(19) United States
(12) Patent Application Publication
(10) Pub. No.: US 2012/0015923 A1
(43) Pub. Date: Jan. 19, 2012

(54) USE OF ANDROGRAPHOLIDE COMPOUNDS FOR TREATING INFLAMMATION AND AIRWAY DISORDERS

(75) Inventor: Wai Shiu Fred Wong, Singapore (SG)

(73) Assignee: NATIONAL UNIVERSITY OF SINGAPORE, Singapore (SG)

(21) Appl. No.: 13/258,894

(22) PCT Filed: Mar. 24, 2010
(86) PCT No.: PCT/SG10/00113

§ 371 Date: Sep. 22, 2011

Related U.S. Application Data
(60) Provisional application No. 61/162,861, filed on Mar. 24, 2009.

(51) Int. Cl. A61K 31/365; A61K 31/573; A61K 31/58; A61K 31/575; C12N 5/071; A61P 11/00; A61P 11/06; A61P 29/00; C07D 307/58; A61K 31/56; C07D 307/60

(52) U.S. Cl. 514/171; 514/473; 549/477; 549/479; 435/375

(57) ABSTRACT

We describe for the first time that andrographolide derivatives such as DDAG effectively reduced OVA-induced inflammatory cell recruitment into BAL fluid, IL-4, IL-5, IL-13 and eotaxin production, serum IgE synthesis, pulmonary eosinophilia, mucus hypersecretion and AHR in a mouse asthma model potentially via inhibition of NF-?B activity. Moreover, low dose of DDAG and glucocorticoid combination treatment synergistically attenuate inflammation in mouse asthma model. These findings support a therapeutic value for DDAG in the treatment of asthma.

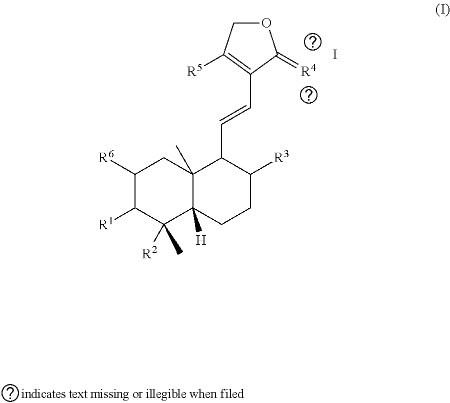

### CROSS-REFERENCE TO RELATED APPLICATION

This application claims benefit of, and priority from, U.S. provisional patent application No. 61/162,861, filed on 24 Mar. 2009, the contents of which are hereby incorporated herein by reference.

### FIELD

The invention relates to compounds for the treatment of airway disorders such as asthma and chronic obstructive pulmonary disease.

### BACKGROUND

Airway disorders such as asthma and chronic obstructive pulmonary disease (COPD) afflict a large number of people. At present, there are about 300 million people worldwide suffering from asthma. It is predicted that the prevalence will go up to 400 million by 2025. Asthma is a common chronic lung disease of the airways that is complex and characterized by variable and recurring symptoms, airflow obstruction, bronchial hyperresponsiveness (bronchospasm), and an underlying inflammation. The interaction of these features of asthma determines the clinical manifestations and severity of asthma and the response to treatment. The rising incidence and prevalence of asthma worldwide have signalled for a need to develop better therapeutic agents. The pathophysiology of asthma is multifactorial, involving a complex network of immune responses and reactions. Furthermore, exposure to uncertained environmental factors can lead to different asthmatic responses in people with dissimilar genetic backgrounds. All these contribute to the uncertainties in asthmatic occurrence, thus making the control of asthma difficult.

Asthma is a chronic airway disorder characterized by airway inflammation, mucus hypersecretion, and airway hyperresponsiveness (AHR)¹. The precise mechanisms that lead to the occurrence of AHR are not fully understood yet, but it is found to be related to the inflammation response mediated by mast cells and eosinophils. Cumulative evidence revealed that these inflammatory responses are mediated by T-helper type 2 (Th2) cells together with mast cells, B cells and eosinophils, as well as a number of inflammatory cytokines and chemokines¹⁻². IL-4 is imperative for B cell isotype switching for the synthesis of immunoglobulin (Ig)E. Allergen-induced crosslinking of IgE-bound high affinity IgE receptors (FcεRI) on the surface of mast cells leads to degranulation and activation of mast cells, and the release of inflammatory mediators like histamine, leukotrienes and cytokines, and immediate broncho-constriction³⁻⁴. IL4 5 is vital for the growth, differentiation, recruitment, and survival of eosinophils which contribute to inflammation and even airway remodeling in asthma⁵. IL-13 plays a pivotal role in the effector phase of Th2 responses such as eosinophilic inflammation, mucus hypersecretion, AHR and airway remodeling⁶. In addition, chemokines such as RANTES (regulated on activation, normal T cells expressed and secreted) and eotaxin are crucial to the delivery of eosinophils to the airways⁷. Airway eosinophilia, together with Th2 cytokines IL-4, IL-5 and IL-13, may ultimately contribute to AHR in asthma⁸. Persistent activation of nuclear factor (NF)-κB has been associated with the development of asthma.

Current therapy for asthma comprises of bronchodilators and anti-inflammatory agents. Currently there are three anti-Inflammatory agents for controlling asthma, which Include 1) Inhaled steroids, 2) cysteinyl-leukotriene receptor antagonist and 3) cromolyn. However, the therapeutic efficacies of cysteinyl-leukotriene receptor antagonist and cromolyn are highly variable and may be limited to certain subgroup of patients. Inhaled corticosteroid, helps to suppress inflammation and reduces the swelling of the lining of the airways, however the glucocorticoid usage is associated with major side effects, and about 5-10% asthmatics are steroid-resistant. Corticosteroid-resistant patients present considerable management problems as there are few alternative anti-inflammatory treatments available⁵⁸.

The first line therapy for the control of mild to severe asthma patients involves the usage of a combination of high-dose inhaled corticosteroids (CS) and long-acting β2-agonists (LABAs). Patients with severe persistent asthma often require additional medications, such as anti-leukotrines and anti-IgE therapies. Glucocorticoid is most commonly used in treating asthma, and inhaled corticosteroids have become established as first-line treatment in patients with persistent asthma²¹. Nonetheless, there is a small proportion (5-10%) of asthmatic patients fail to respond to glucocorticoid even at high doses or with supplementary therapy²². Moreover, there has been increasing concern over the side effects of CS such as osteoporosis, glaucoma, weight gain, reduced bone density, muscle breakdown, anovulation, growth retardation in children and poor wound healing effects¹⁵. As prescribing a higher dose of LABA could lead to the occurrence of undesirable side effects, it is thus generally recommended that LABA should be used together with CS or theophylline for better treatment. Although the combination of CS and LABA is by far the most successful treatment used in treating asthma, the occurrence of adverse effects make it necessary to have alternatives treatments for asthma.

Chronic obstructive pulmonary disease (COPD) refers to chronic bronchitis and emphysema, two commonly co-existing diseases of the lungs in which the airways become narrowed (14). This leads to a limitation of the flow of air to and from the lungs causing shortness of breath. In contrast to asthma, the limitation of airflow is poorly reversible and usually gets progressively worse over time.

COPD is caused by noxious particles or gas, most commonly from tobacco smoking, which triggers an abnormal inflammatory response in the lung. The natural course of COPD is characterized by occasional sudden worsening of symptoms called acute exacerbations, most of which are caused by infections or air pollution. COPD is also known as chronic obstructive lung disease (COLD), chronic obstructive airway disease (COAD), chronic airflow limitation (CAL) and chronic obstructive respiratory disease (CORD).

There is currently no cure for COPD and the only measures that have been shown to reduce mortality are smoking cessation and supplemental oxygen (14). COPD can be managed with bronchodilators such as β₂ agonists and/or anticholinergics. β₂ agonist stimulate β₂ receptors while anticholinergics block stimulation from cholinergic nerves both are medicines that relax smooth muscle around the airways, increasing air flow. There are several β₂ agonists available, salbutamol or albuterol and terbutaline are widely used short acting β₂ agonists and provide rapid relief of COPD symptoms. Long acting β₂ agonists (LABAs) such as salmeterol and formoterol are used as maintenance therapy. Ipratropium is the most widely prescribed short acting anticholinergic drug. Anticholinergics appear to be superior to β₃₂ agonists in COPD, however both β₂ agonists and anticholinergics do not have anti-inflammatory actions and they do not halt progression of COPD.

Andrographolide is a labdane diterpenoid that is the main bioactive component of the medicinal plant Andrographis paniculata (Burm. f.) Nees, (Acanthaceae). Andrographolide is an extremely bitter substance extracted from the stem and leaves of the Andrographis paniculata. The plant is grown for medicinal purposes in China and India and has traditionally been used as herbal medicine for common cold, fever and non-infectious diarrhea. Andrographolide has been shown to be effective against certain cancers and has also been shown to possess anticancer⁹⁻¹⁰, and hepatocyte-protective activities¹¹.

14-Deoxy-11,12-didehydroandrographolide (DDAG) C₂₀H₂₈O₄, is another diterpenoid isolated from A. paniculata¹⁸⁻¹⁹ . The structure of andrographolide and 14-deoxy-1 1,12-didehydroandrogapholide (DDA) is depicted In FIG. 1.

### SUMMARY

Accordingly, a first aspect of the invention comprises a method of controlling inflammation in a lung cell comprising administering a dose of formula I.

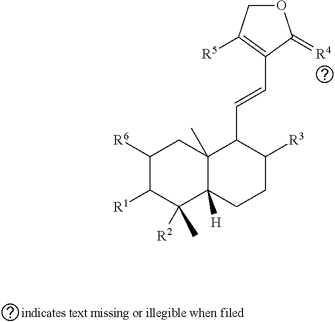

wherein,
R¹ and R² may be selected from a hydroxyl group, a methoxy group, a methylene group, or an ether or ester linked sugar group; hydrogen, substituted or unsubstituted, linear or branched (C₁-C₈) alkyl group such as methyl, ethyl, n-propyl, iso-propyl and the like; aryl group such as phenyl, naphthyl and the like, the aryl group may be substituted; heteroaryl group such as pyridyl, furyl, thiophenyl and the like, the heteroaryl group may be substituted; aralkyl such as benzyl, phenethyl and the like, the aralkyl group may be substituted; heteroaralkyl group such as pyridylmethyl, pyridylethyl, furanmethyl, faranethyl and the like, the heteroaralkyl group may be substituted; (C₂-C₈) alkanoyl group such as ethanoyl, propanoyl, butanoyl and the like, the (C₂-C₈) alkanoyl group may be substituted; (C₃-C₈) alkenoyl group such as propenoyl, butenoyl, pentenyl and the like, (C₃-C₈) alkenoyl group may be substituted; aroyl group such as benzoyl and the like, the aroyl group may be substituted; heteroaroyl group such as pyridyl carbonyl, furyl carbonyl and the like; the heteroaroyl group may be substituted; aralkenoyl group such as phenylpropenoyl, phenylbutenoyl, phenylpentenoyl and the like, the aralkenoyl group may be substituted; aralkanoyl group such as phenylpropanoyl, phenylbutanoyl, phenylpentanoyl and the like, the aralkanoyl group may be substituted; sulfonyl group such as methanesulfonyl, benzenesulfonyl, p-toluenesulfonyl and the like, the sulfonyl group may be substituted.
R³ is selected from a methyl group or a methylene group;
R⁴ is selected from a hydroxyl group or a carbonyl group;
R⁵ is selected from one of the following: a hydroxyl group, an alkyl group, a methoxy group, a methylene group, or an ether or ester linked sugar group;

In one embodiment the cell is in vitro. In another embodiment the cell is in vivo and the formula I is administered to a patient in need of controlling an airway disorder.

In one embodiment formula I is Andrographolide. In another embodiment formula I is 14-deoxy-1 1,12-didehydroandrographolide.

In one embodiment controlling inflammation comprises controlling asthma. In another embodiment controlling inflammation comprises controlling allergenic effects. In another embodiment controlling inflammation comprises controlling chronic obstructive pulmonary disease (COPD).

Another aspect of the invention comprises a method of treating an airway disorder comprising administering a dose of formula I as defined above. In one embodiment formula I is Andrographolide for treating an airway disorder. In another embodiment formula I is 14-deoxy-1 1,12-didehydroandrographolide for treating an airway disorder. In one embodiment the airway disorder is an asthma exacerbation. In another embodiment the airway disorder is COPD. In another embodiment the method of treating the airway disorder may further comprising administering a corticosteroid.

Another aspect of the invention comprises a compound of formula I as defined above for use in treating an airway disorder. In one embodiment formula I is Andrographolide. In another embodiment formula I is 14-deoxy-1 1,12-didehydroandrographolide. In one embodiment the compound may be for treating the airway disorder of an asthma exacerbation. In another embodiment the compound may be for treating the airway disorder of COPD. In another embodiment the compound may further comprising administering a corticosteroid.

Another aspect of the invention comprises a Composition of a corticosteroid and formula I as described above. In one embodiment formula I of the composition may be Andrographolide. In another embodiment formula I of the composition may be 14-deoxy-1 1,12-didehydroandrographolide. In another embodiment the corticosteroid of the composition may be Dexamethasone, Budesonide, Fluticasone, Ciclesonide, or Beclomethasone Dipropionate.

[019]. In one embodiment the Composition may be for use in treating airway disorders such asthma or COPD.

### BRIEF DESCRIPTION OF THE DRAWINGS

The invention will be better understood by reference to the following description of several specific embodiments thereof as shown in the accompanying drawings in which:

FIG. 1: Chemical depiction of the structures of (A) andrographolide and (B) 14-deoxy-1 1,12-didehydroandrographolide (DDAG).

FIG. 2: Effects of andrographolide on OVA-induced inflammatory cell recruitment and mucus hypersecretion. (A) Inflammatory cell counts in BAL fluid obtained from sensitized mice 24 hours after the last saline (n=6 mice per group) or OVA (n=7 mice per group) aerosol challenge. Andrographolide dose-dependently reduced OVA-induced inflammatory cell counts in BAL fluid from sensitized mice 24 hours after the last OVA aerosol challenge (DMSO, n=7; 0.1 mg/kg, n=7; 0.5 mg/kg, n−10; and 1 mg/kg, n=9 mice per group). Differential cell counts were performed on a minimum of 500 cells to identify eosinophll (Eos), macrophage (Mac), neutrophil (Neu), and lymphocyte (Lym). Histological examination of lung tissue eosinophilla (H&E, magnification×200) and mucus secretion (PAS, magnification×200) 24 hours after the last challenge of saline aerosol, OVA aerosol, OVA aerosol plus DMSO, or OVA aerosol plus 1 mg/kg andrographolide. *Significant difference from DMSO control, P<0.05.

FIG. 3. Effects of andrographolide on cytokine levels In BALF. BAL fluids were collected 24 hours after the last OVA aerosol challenge. Level of IL-4, IL-5, IL-13 and IFN-γy were analysed using enzyme-linked Immunosorbant assay (ELISA).

FIG. 4. Effects of andrographolide on serum IgE production. Mouse serum was collected 24 hours after the last OVA aerosol challenge. The levels of OVA-specific IgE and total IgE were analysed using ELISA. Andrographolide significantly lowered total IgE and OVA-specific IgE levels, Indicating an OVA-specific Inhibition on the Th2 response by andrographolide

FIG. 5. Effects of andrographolide on pulmonary mRNA expression of Inflammatory markers. Lung tissues were collected 24 hours after the last OVA aerosol challenge. Total mRNA was extracted using TriZol reagent and the PCR product were separated in a 2% agarose gel visualized under UV light. β-actin was used as an internal control.

FIG. 6. Effects of andrographolide on OVA-induced airway hyper-responsiveness. Airway responsiveness of mechanically ventilated mice in response to intravenous methacholine was measured 24 hours after the last saline aerosol or OVA aerosol with pre-treatment of either DMSO or 1 mg/kg andrographolide. AHR is expressed as percentage change from the baseline level of (A) lung resistance (RI, n=5 mice per treatment group) and (B) dynamic compliance (Cdyn, n=5 mice per treatment group). RI Is defined as the pressure driving respiration divided by flow. Cdyn refers to the distensibility of the lung and is defined as the change in volume of the lung produced by a change in pressure across the lung.

FIG. 7. (A) Effects of andrographolide on TNF-α stimulation of normal human bronchial epithelial cells. Epithelial cells were stimulated with 10 ng/ml TNF-α in the presence and absence of 30 μM andrographolide for 5, 15 and 30 minutes before total proteins were extracted for subsequent immunoblotting analysis Immunoblots were probed with anti-IKKβ, anti-phospho-IKKβ (Ser¹⁸⁰), anti-IκBα, anti-phospho-IκBα (Ser^{32/36}), anti-p65, anti-phospho-p65 (Ser⁵³⁶) or anti-β-actin antibody, and developed by enhanced chemiluminescence reagent. (B) Immunoblotting of p65 level in nuclear extracts of epithelial cells stimulated with TNF-α for 30 minutes in the presence and absence of 30 μM andrographolide. Nuclear proteins were separated by 10% SDS-PAGE, probed with anti-p65 or anti-TBP antibody, and quantitated using Gel-Pro imaging software. TBP nuclear protein was used as an internal control. DNA-binding activity of p65 NF-κB in nuclear extracts of epithelial cells stimulated with TNF-α a for 30 minutes in the presence and absence of 30 μM andrographolide was determined using a TransAM™ p65 transcription factor ELISA kit.

FIG. 8. Effects of DDA on OVA-induced inflammatory cell recruitment and mucus hypersecretion. In the mouse asthma model using ovalbumin as aeroallergen, we showed that DDA dose-dependently inhibited ovalbumin-induced cell Infiltration Into the airways obtained from bronchoalveolar lavage fluid (A) Inflammatory cell counts in BAL fluid obtained from sensitized mice 24 hours after the last saline or OVA aerosol challenge. DDA dose-dependently reduced OVA-induced inflammatory cell counts in BAL fluid from sensitized mice 24 hours after the last OVA aerosol challenge. Differential cell counts were performed on a minimum of 500 cells to Identify eosinophil (Eos), macrophage (Mac), neutrophil (Neu), and lymphocyte (Lym). (B) Histological examination of lung tissue eosinophilla (H&E, magnification×200) and mucus secretion (PAS, magnification×200) 24 hours after the last challenge of saline aerosol (OS), OVA aerosol (OO), OVA aerosol plus DMSO (DMSO), or OVA aerosol plus 1 mg/kg DDA (DDA).

FIG. 9. Effects of DDAG on OVA-induced inflammatory cell recruitment and mucus hypersecretion. (A) Inflammatory cell counts in BAL fluid obtained from sensitized mice 24 hours after the last saline aerosol (n=7 mice per group) or OVA aerosol (n=7 mice per group) challenge. DDAG dose-dependently reduced OVA induced inflammatory cell counts in BAL fluid from sensitized mice 24 hours after the last OVA aerosol challenge (DMSO, n=7; 0.1 mg/kg, n=8; 0.5 mg/kg, n=7; and 1 mg/kg, n=10 mice per group). Differential cell counts were performed on a minimum of 500 cells to identify eosinophil (Eos), macrophage (Mac), neutrophil (Neu), and lymphocyte (Lym). Histological examination of lung tissue eosinophilia (B), (magnification×200) and mucus secretion (C), (magnification×200) 24 hours after the last challenge of saline aerosol, OVA aerosol, OVA aerosol plus DMSO, or OVA aerosol plus 1 mg/kg DDAG. Quantitative analyses of inflammatory cell infiltration and mucus production in lung sections were performed. Briefly, to determine the severity of inflammatory cell infiltration, peribronchial cell counts were performed. To determine the extent of mucus production, goblet cell hyperplasia in the airway epithelium was quantified blind using a 5-point grading system. *Significant difference from DMSO control, P<0.05.

FIG. 10. Mast cells were detected in lung tissue using toluidine-blue staining (A). The number of degranulating and intact mast cells was counted in paraffin sections. The percentage of degranulated mast cells in the lung was calculated by counting the number of cells with 10% of extrusion of granules (B).

FIG. 11. Effects of DDAG on OVA-induced BAL fluid cytokine and chemokine levels and serum Ig production. (A) BAL fluids were collected 24 hours after the last OVA aerosol challenge. Levels of IL-4, IL-5, IL-13, eotaxin and IFN-γ were analyzed using ELISA (n=6-9 mice per group). Lower limits of detection were as follows: IL-1 and IL-5 at 4 pg/ml; IL-13 and IFN-γ at 15.6 pg/ml; and eotaxin at 2 pg/ml. (B) Mouse serum was collected 24 hours after the last OVA aerosol challenge. The levels of total IgE, OVA-specific IgE, OVA-specific IgG1, and OVA-specific IgG2a were analyzed using ELISA (n=6-9 mice per group). Values shown are the mean±SEM. *Significant difference from DMSO control, P<0.05.

FIG. 12: Effects of DDAG on OVA-induced AHR. Airway responsiveness of mechanically ventilated mice in response to intravenous methacholine was measured 24 hours after the last saline aerosol or OVA aerosol with pretreatment of either DMSO or 1 mg/kg DDAG. AHR is expressed as percentage change from the baseline level of (A) lung resistance (R1, n=7-9 mice per treatment group) and (B) dynamic compliance (Cdyn, n=7-9 mice per treatment group). RI is defined as the pressure driving respiration divided by flow. Cdyn refers to the distensibility of the lung and is defined as the change in volume of the lung produced by a change in pressure across the lung. *Significant difference from DMSO control, P<0.05. (C)

FIG. 13: Effects of DDAG on OVA-induced NF-KB activity and inflammatory gene expression in allergic airway inflammation. Lung tissues were collected 24 hours after the last OVA aerosol challenge. Total mRNA was extracted using TriZol reagent and the PCR products were separated in a 2% agarose gel visualized under UV light. β-actin was used as an internal control. The experiments were repeated for three times (n=3 mice per group) with similar pattern of results.

FIG. 14: Immunoblotting of p65 NF-κB in nuclear extract of lung tissues (A) isolated from mice 24 hours after the last saline aerosol or OVA aerosol challenge pretreated with either DMSO or 1 mg/kg DDAG or immunoblotting of p65 NF-κB in nuclear extract of normal human bronchial epithelial cells (C) stimulated with 10 ng/ml TNF-α in the presence and absence of 30 μM DDAG for 5 minutes. Nuclear proteins were separated by 10% SDS-PAGE, probed with anti-p65 or anti-TBP antibody, and developed by enhanced chemiluminescence reagent. TBP nuclear protein was used as an internal control. The experiments were repeated for three times (n=3 mice per group) with similar pattern of results. Nuclear p65 DNA-binding activity of nuclear extract of both lung tissues and nuclear extracts of epithelial cells stimulated with TNF-α for 5 minutes in the presence and absence of 30 μM DDAG was determined using a TransAM p65 transcription factor ELISA kit. Values shown are the mean±SEM of three separate experiments. *Significant difference from DMSO control, P<0.05. (E) Epithelial cells were stimulated with 10 ng/ml TNF-α in the presence and absence of 30 μM DDAG for 12 hours before total mRNA was extracted using TriZol reagent. PCR products were separated in a 2% agarose gel and visualized under UV light. β-actin was used as an internal control. This is a representative gel from 3 separate experiments with similar pattern of results. Values shown are the mean±SEM of three separate experiments. *Significant difference from DMSO control, P<0.05.

FIG. 15: Effects of DDAG and Glucocorticoid (Dexamathasone, Dex) independently or in combination on OVA-induced inflammatory cell recruitment. (A) Inflammatory cell counts in BAL fluid obtained from sensitized mice 24 hours after the last OVA aerosol (n=4 mice per group) challenge. Low dose of DDAG (0.1 mg/kg) and low dose of Dexamethasone (0.05 mg/kg) significantly reduced OVAinduced inflammatory cell counts in BAL fluid from sensitized mice 24 hours after the last OVA aerosol challenge. Differential cell counts were performed on a minimum of 500 cells to identify eosinophil (Eos), macrophage (Mac), neutrophil (Neu), and lymphocyte (Lym). Effects of DDAG on OVA-induced BAL fluid cytokine and chemokine levels and serum Ig production. (A) BAL fluids were collected 24 hours after the last OVA aerosol challenge. Levels of IL-4, IL-5, IL-13, and Eotaxin were analyzed using ELISA (n=6-9 mice per group). Values shown are the mean±SEM. *Significant difference from DMSO control, P<0.05.

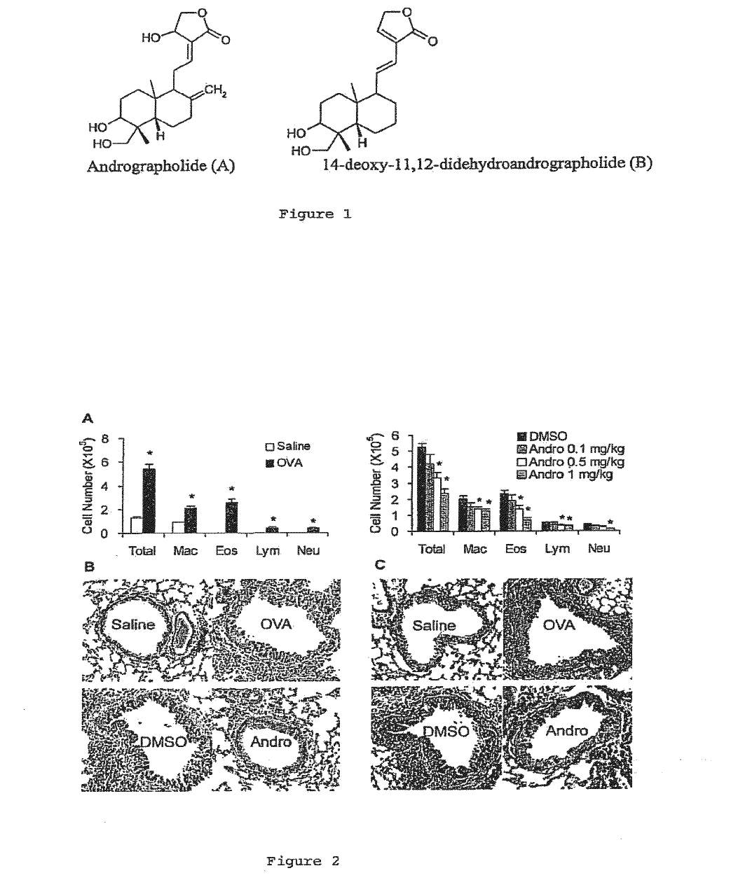

### DETAILED DESCRIPTION

Andrographis paniculata and/or andrographolide compounds were used to effectively reduced OVA-induced inflammatory cell recruitment into BAL fluid, IL-4, IL-5, IL-13 and eotaxin production, serum IgE synthesis, pulmonary eosinophilia, mucus hypersecretion and AHR in a mouse asthma model.

### Compounds of the Invention

“Compounds” include known andrographolide compounds wherein the compound has the following structure:

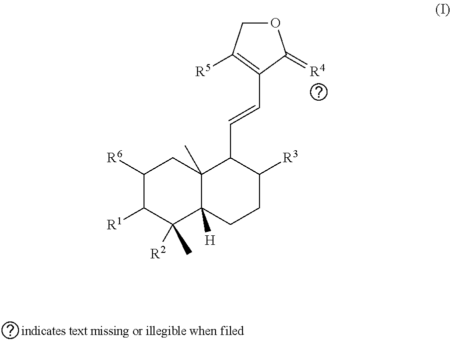

wherein,
R′ and R² may be selected from a hydroxyl group, a methoxy group, a methylene group, or an ether or ester linked sugar group; hydrogen, substituted or unsubstituted, linear or branched (C₁-C₈) alkyl group such as methyl, ethyl, n-propyl, iso-propyl and the like; aryl group such as phenyl, naphthyl and the like, the aryl group may be substituted; heteroaryl group such as pyridyl, furyl, thiophenyl and the like, the heteroaryl group may be substituted; aralkyl such as benzyl, phenethyl and the like, the aralkyl group may be substituted; heteroaralkyl group such as pyridylmethyl, pyridylethyl, furanmethyl, faranethyl and the like, the heteroaralkyl group may be substituted; (C₂-C₈) alkanoyl group such as ethanoyl, propanoyl, butanoyl and the like, the (C₂-C₈) alkanoyl group may be substituted; (C₃-C₈) alkenoyl group such as propenoyl, butenoyl, pentenyl and the like, (C₃-C₈) alkenoyl group may be substituted; aroyl group such as benzoyl and the like, the aroyl group may be substituted; heteroaroyl group such as pyridyl carbonyl, furyl carbonyl and the like; the heteroaroyl group may be substituted; aralkenoyl group such as phenylpropenoyl, phenylbutenoyl, phenylpentenoyl and the like, the aralkenoyl group may be substituted; aralkanoyl group such as phenylpropanoyl, phenylbutanoyl, phenylpentanoyl and the like, the aralkanoyl group may be substituted; sulfonyl group such as methanesulfonyl, benzenesulfonyl, p-toluenesulfonyl and the like, the sulfonyl group may be substituted.

Suitable cyclic structures formed by OR² and OR³ may be selected from-0-(CR⁷R⁸)ₘ-O-where R⁷ and R⁸ may be same or different and independently represent hydrogen, unsubstituted or substituted groups selected from (C₁-C₆) alkyl such as methyl, ethyl, n-propyl and the like; aryl group such as phenyl, naphthyl and the like, the aryl group may be substituted; heteroaryl group such as pyridyl, furyl, thiophenyl, pyrrolyl and the like; the heteroaryl group may be substituted or R⁷ and R⁸ together represent ‘C=0’; m represents an integer 1 or 2. The substituents on R⁷ and R⁸ include hydroxy, halogen such as fluorine, chlorine, bromine and the like; nitro, cyano or amino groups.

The substituents on R² may be selected from cyano, hydroxy, nitro, thio, halogen atom such as fluorine, chlorine, bromine and the like; substituted or unsubstituted groups selected from linear or branched (C₁-C₈) alkyl group such as methyl, ethyl, n-propyl, iso-propyl and the like; amino, mono or disubstituted amino group; alkanoyl group such as ethanoyl, propanoyl, butanoyl and the like; thio (C₁-C₈) alkyl such as thiomethyl, thioethyl, thiopropyl and the like; (C₁-C₆) alkoxy group such as methoxy, ethoxy, propyloxy, butyloxy and the like; aroyl group such as benzoyl and the like; acyloxy group such as acetyloxy, propanoyloxy, butanoyloxy and the like; aryl group such as phenyl, naphthyl and the like, the aryl group may be mono or disubstituted; heteroaryl group such as pyridyl, furyl, thienyl and the like; acylamino groups such as CH₃CONH, C₂H₅CONH, C₃H₇CONH, C₄H₉CONH and C₆H₅CONH; aralkylamino group such as C₆H₅CH₂NH, C₆H₅CH₂CH₂NH, C₆H₅CH₂NCH₃ and the like; alkoxycarbonylamino group such as C₄H₉OCONH, C₂H₅OCONH, CH₃OCONH and the like; aryloxycarbonylamino group such as C₆H₅0CONH, C₆H₅OCONCH₃, C₆H₅OCONC₂H₅, C₆H₄ (CH₃) OCONH, C₆H₄ (OCH₃) OCONH and the like; aralkoxycarbonylamino group such as C₆H₅CH₂OCONH, C₆H₅CH₂CH₂OCONH, C₆H₅CH₂OCON (CH₃), C₆H₅CH₂OCON (C₂H₅), C₆H₄ (CH₃) CH₂OCONH, C₆H₄ (OCH₃) CH₂OCONH and the like; (C₁-C₈) alkylthio group such as methylthio, ethylthio, propylthio and the like; heteroarylthio group such as pyridylthio, furylthio, thiophenylthio, benzothiazolethio, purinethio, benzimidazolethio, pyrimidinethio and the like; acylthio group such as acetylthio, propanoylthio, butanoylthio and the like; aralkylthio group such asbenzylthio, phenylethylthio, phenylpropylthio and the like; arylthio group such as phenylthio, napthylthio and the like; (C₁-C₈) alkylseleno such as methylseleno, ethylseleno, propylseleno, iso-propylseleno and the like; acylseleno such as acetylseleno, propionylseleno and the like; aralkylseleno such as benzylseleno, phenylethylseleno, phenylpropylseleno and the like; arylseleno such as phenylseleno, napthylseleno and the like or COOR, where R represents hydrogen or (C₁-C₆) alkyl groups. The substituents are selected from halogen, hydroxy, nitro, cyano, amino, (C₁-C₆) alkyl, aryl or (C₁-C₆) alkoxy groups.

When the groups R² represent disubstituted aryl, the two substituents on the adjacent carbon atoms form a linking group such as-X—CH₂—Y—, —X—CH₂—CH₂—Y—, where X and Y may be same or different and independently represent O, NH, S or CH₂ When the groups represented by R² are multi substituted, the substituents present on the two adjacent carbons may form a linking group X— (CR⁹R¹⁰)ₙ_{_{—}}Y_where R⁷ and R⁸ represent (C₁-C₅) alkyl such as methyl, ethyl and the like, X and Y may be same or different and independently represent CH₂, O, S, NH; and n=1 or 2.
R³ is selected from a methyl group or a methylene group;
R⁴ is selected from a hydroxyl group or a carbonyl group;
R⁵ is selected from one of the following: a hydroxyl group, an alkyl group, a methoxy group, a methylene group, or an ether or ester linked sugar group;
The formula includes the following naturally occurring analogs: 14-epiandrographolide; isoandrographolide; 14-deoxy-12-methoxyandrographolide; 12-epi-14-12-methσxyandrogapholide; 14-deoxy-12-hydroxyandrographolide; and 14-deoxy-1 1-hydroxyandrographolide. The formula further includes derivatives of andrographolide.

[026]. Pharmaceutically acceptable salts forming part of this invention include salts derived from inorganic bases such as Li, Na, K, Ca, Mg, Fe, Cu, Zn, Mn; salts of organic bases such as N, N′-diacetylethylenediamine, betaine, caffeine, 2-diethylamino ethanol, 2-dimethylaminoethanol, N-ethylmorpholine, N-ethylpiperidine, glucamine, glucosamine, hydrab amine, isopropylamine, methylglucamine, morpholine, piperazine, piperidine, procaine, purines, theobromine, triethylamine, trimethylamine, tripropylamine, tromethamine, diethanolamine, meglumine, ethylenediamine, N, N′-diphenylethylenediamine, N, N′-dibenzylethylenediamine, N-benzyl phenylethylamine, choline, choline hydroxide, dicyclohexylamine, metfonnin, benzylamine, phenylethylamine, dialkylamine, trialkylamine, thiamine, aminopyrimidine, aminopyridine, purine, spermidine, and the like; chiral bases like alkylphenylamine, glycinol, phenyl glycinol and the like, salts of natural amino acids such as glycine, alanine, valine, leucine, isoleucine, norleucine, tyrosine, cystine, cysteine, methionine, proline, hydroxy proline, histidine, omithine, lysine, arginin, serine, threonine, phenylalanine; unnatural amino acids such as D-isomers or substituted amino acids; guanidine, substituted guanidine wherein the substituents are selected from nitro, amino, alkyl, alkenyl, alkynyl, ammonium or substituted ammonium salts and aluminum salts.

Salts may include acid addition salts where appropriate which are, sulphates, nitrates, phosphates, perchlorates, borates, hydrohalides, acetates, tartrates, maleates, citrates, succinates, palmoates, methanesulphonates, benzoates, salicylates, hydroxynaphthoates, benzenesulfonates, ascorbates, glycerophosphates, ketoglutarates and the like.

Pharmaceutically acceptable solvates may be hydrates or comprising other solvents of crystallization such as alcohols.

Particularly useful compounds of the present invention include: 3, 1 9-Diacetyl-12-(N-benzylamino)-14-deoxy andrographolide; 3,19-Diacetyl-12α-(N-benzylamino)-14-deoxy andrographolide; 3,19-Diacetyl-12 β-(N-benzylamino)-14-deoxy andrographolide; 14-Deoxy-12-(O-methylphenylglycino)-3, 19-O-(1-phenylethylidene) andrographolide; 14-Deoxy-12α-(O-methylphenylglycino)-3, 19-0-(I-phenylethylidene) andrographolide; 14-Deoxy-12β-(O-methylphenylglycino)-3, 19-0-(1-phenylethylidene) andrographolide; 3,19-Diacetyl-14-deoxy-12-(N-4-methoxybenzylamino) andrographolide; 3,19-Diacetyl-14-deoxy-12α-(N-4-methoxybenzylamino) andrographolide; 3,19-Diacetyl-14-deoxy-12β-(N-4-methoxybenzylamino) andrographolide; 3,19-Diacetyl-12-(N-2-chlorobenzylamino)-14-deoxy andrographolide; 3,19-Diacetyl-12α-(N-2-chlorobenzylamino)-14-deoxy andrographolide; 3,19-Diacetyl-12β-(N-2-chlorobenzylamino)-14-deoxy andrographolide; 3,19-Diacetyl-14-deoxy-12-(O-methylprolino) andrographolide; 3,19-Diacetyl-14-deoxy-12α-(O-methylprolino) andrographolide; 3,19-Diacetyl-14-deoxy-12β-(O-methylprolino) andrographolide; 3,19-Diacetyl-14-deoxy-12-(O-methylphenylalanino) andrographolide; 3,19-Diacetyl-14-deoxy-12α-(O-methylphenylalanino) andrographolide; 3,19-Diacetyl-14-deoxy-12β-(O-methylphenylalanino) andrographolide; 3,19-Diacetyl-14-deoxy-12-(O-methyl-3-phenylisoserino) andrographolide; 3,19-Diacetyl-14-deoxy-12α-(O-methyl-3-phenylisosetino) andrographolide; 3,19-Diacetyl-14-deoxy-12β-(O-methyl-3-phenylisoserino) andrographolide; 3,19-Diacetyl-14-deoxy-12-(O-methylmethionino) andrographolide; 3,19-Diacetyl-14-deoxy-12α-(O-methylmethionino) andrographolide; 3,19-Diacetyl-14-deoxy-12β-(O-methylmethionino) andrographolide; 3,19-Diacetyl-14-deoxy-12-(O-methylphenylglycino) andrographolide; 3,19-Diacetyl-14-deoxy-12αa-(O-methylphenylglycino) andrographolide; 3,19-Diacetyl-14-deoxy-12β-(O-methylphenylglycino) andrographolide; 3,19-Diacetyl-14-deoxy-12-(O-methylalanino) andrographolide; 3,19-Diacetyl-14-deoxy-12α-(O-methylalanino) andrographolide; 3,19-Diacetyl-14-deoxy-12β-(O-methylalanino) andrographolide; 3,19-Diacetyl-14-deoxy-12-(O-methylglycino) andrographolide; 3,19-Diacetyl-14-deoxy-12α-(O-methylglycino) andrographolide; 3,19-Diacetyl-14-deoxy-12β-(O-methylglycino) andrographolide; 3,19-Diacetyl-14-deoxy-12-(O-methylselenomethionino) andrographolide; 3,19-Diacetyl-14-deoxy-12α-(O-methylselenomethionino) andrographolide; 3,19-Diacetyl-14-deoxy-12β-(O-methylselenomethionino) andrographolide; 3,19-Diacetyl-14-deoxy-12-(N-imidazolyl) andrographolide; 3,19-Diacetyl-14-deoxy-12α-(N-imidazolyl) andrographolide; 3,19-Diacetyl-14-deoxy-12β-(N-imidazolyl) andrographolide; 3,19-Diacetyl-14-deoxy-12-(N-methylpiperazino) andrographolide; 3,19-Diacetyl-14-deoxy-12α-(N-methylpiperazino) andrographolide; 3,19-Diacetyl-14-deoxy-12β(N-methylpiperazino) andrographolide; 3,19-Diacetyl-14-deoxy-12-morpholino andrographolide; 3,19-Diacetyl-14-deoxy-12α-morpholino andrographolide; 3,19-Diacetyl-14-deoxy-12β-morpholino andrographolide, 3,19-Diacetyl-12-(N-acetylpiperazino)-14-deoxy andrographolide; 3,19-Diacetyl-12α-(N-acetylpiperazino)-14-deoxy andrographolide; 3,19-Diacetyl-12p-(N-acetylpiperazino)-14-deoxy andrographolide; 12-(N-Benzylamino)-14-deoxy andrographolide; 12α-(N-Benzylamino)-14-deoxy andrographolide; 12β-(N-Benzylamino)-14-deoxy andrographolide; 14-Deoxy-12-(O-methylphenylglycino) andrographolide; 14-Deoxy-12α-(O-methylphenylglycino) andrographolide; 14-Deoxy-12β-(O-methylphenylglycino) andrographolide; 14-Deoxy-3, 19-0-isopropylidene-12-(methylphenylalanino) andrographolide; 14-Deoxy-3,19-0-isopropylidene-12α-(methylphenylalanino) andrographolide; 14-Deoxy-3,19-0-isopropylidene-12β(methylphenylalanino) andrographolide; 12-(N-Benzylamino)-14-deoxy-3, 19-O-(1-phenylethylidene) andrographolide; 12α-(N-Benzylamino)-14-deoxy-3, 19-O-(1-phenylethylidene) andrographolide; 12β-(N-Benzylamino)-14-deoxy-3, 19-0-(1-phenylethylidene) andrographolide; 14-Deoxy-12-(O-methylphenylalanino)-3, 19-0-(1-phenylethylidene) andrographolide; 14-Deoxy-12α-(O-methlphenylalanino)-3, 19-O-(1-phenyletliylidene) andrographolide; 14-Deoxy-12β-(O-methylphenylalanino)-3, 19-O-(1-phenylethylidene) andrographolide; 14-Deoxy-12-(O-methylprolino)-3, 19-0-(1-phenylethylidene) andrographolide; 14-Deoxy-12α-(O-methylprolino)-3, 19-0-(1-phenylethylidene) andrographolide; 14-Deoxy-12β-(O-methylprolino)-3, 19-O-(1-phenylethylidene) andrographolide; 3, 19-O-B enzylidene-12-(N-benzylamino)-14-deoxy andrographolide; 3,19-O-Benzylidene-12αa-(N-benzylamino)-14-deoxy andrographolide; 3,19-0-Benzylidene-12β-(N-benzylamino)-14-deoxy andrographolide; 3,19-Diacetyl-14-deoxy-8, 17-epoxy-12-(O-methylmethionino) andrographolide; 3,19-Diacetyl-14-deoxy-8, 17-epoxy-12α-(O-methylmethionino) andrographolide; 3,19-Diacetyl-14-deoxy-8, 17-epoxy-12β-(O-methylmethionino) androgapholide; 3,19-Diacetyl-14-deoxy-8, 17-epoxy-12-(O-methylphenylglycino) andrographolide; 3,19-Diacetyl-14-deoxy-8, 17-epoxy-12α-(O-methylphenylglycino) andrographolide; 3,19-Diacetyl-14-deoxy-8, 17-epoxy-12β-(O-methylphenylglycino) andrographolide; 3,19-Diacetyl-14-deoxy-12-(N-1, 2, 4-triazolyl) andrographolide; 3,19-Diacetyl-14-deoxy-12α-(N-1, 2,4-triazolyl) andrographolide; 3,19-Diacetyl-14-deoxy-12β(N-1,2,4-triazolyl) andrographolide; 14-Deoxy-12-(2, 3-dimethylanilino) andrographolide; 14-Deoxy-12α-(2, 3-dimethylanilino) andrographolide; 14-Deoxy-12β-(2, 3-dimethylanilino) andrographolide; 3,19-Diacetyl-14-deoxy-12-(4-methoxy-2-methylanilino) andrographolide; 3,19-Diacetyl-14-deoxy-12α-(4-methoxy-2-methylanilino) andrographolide; 3,19-Diacetyl-14-deoxy-12β-(4-methoxy-2-inethylanilino) andrographolide; 3,19-Diacetyl-14-deoxy-12-(4-hydroxy-2-methylanilino) andrographolide; 3,19-Diacetyl-14-deoxy-12α-(4-hydroxy-2-methylanilino) andrographolide; 3,19-Diacetyl-14-deoxy-12-(4-hydroxy-2-methylanilino) andrographolide; 3,19-Diacetyl-14-deoxy-12-(2-mercaptoanilino) andrographolide; 3,19-Diacetyl-14-deoxy-12α-(2-mercaptoanilino) andrographolide; 3,19-Diacetyl-14-deoxy-12β-(2-mercaptoanilino) andrographolide; 3,19-Diacetyl-14-deoxy-12-(3, 4-dimethoxyanilino) andrographolide; 3,19-Diacetyl-14-deoxy-12α-(3, 4-dimethoxyanilino) andrographolide; 3,1 9-Diacetyl-14-deoxy-12β-(3, 4-dimethoxyanilino) andrographolide; 3,19-Diacetyl-12-anilino-14-deoxy andrographolide; 3,19-Diacetyl-12α-anilino-14-deoxy andrographolide; 3,1 9-Diacetyl-12β-anilino-14-deoxy andrographolide; 3,19-Diacetyl-14-deoxy-12-(2, 3-dimethylanilino) andrographolide; 3, 19-Diacetyl-14-deoxy-12α-(2, 3-dimethylanilino) andrographolide; 3,19-Diacetyl-14-deoxy-12β-(2, 3-dimethylanilino) andrographolide; 3,19-Diacetyl-14-deoxy-12-(2-methyl-4-methylsulfonateanilino) andrographolide; 3,19-Diacetyl-14-deoxy-12α-(2-methyl-4-methylsulfonateanilino) andrographolide; 3,19-Diacetyl-14-deoxy-12β-(2-methyl-4-methylsulfonateanilino) andrographolide; 3,19-Diacetyl-14-deoxy-12-(N-tetrazolylamino) andrographolide; 3,19-Diacetyl-14-deoxy-12α-(N-tetrazolylamino) andrographolide; 3,19-Diacetyl-14-deoxy-12β-(N-tetrazolylamino) andrographolide; 14-Deoxy-12-(3, 4-dimethoxyanilino) andrographolide; 14-Deoxy-12α-(3, 4-dimethoxyanilino) andrographolide; 14-Deoxy-12β-(3, 4-dimethoxyanilino) androgapholide; 14-Deoxy-3, 19-O-isopropylidene-12-(2, 3-dimethylanilino) andrographolide; 14-Deoxy-3, 19-O-isopropylidene-12α-(2, 3-dimethylanilino) andrographolide; 14-Deoxy-3, 19-0-isopropylidene-12β-(2, 3-dimethylanilino) andrographolide; 14-Deoxy-12-(2-methylanilino)-3, 19-0-(1-phenylethylidene) andrographolide; 14-Deoxy-12α-(2-methylanilino)-3, 19-0-(1-phenylethylidene) andrographolide; 14-Deoxy-12β-(2-methylanilino)-3, 19-O-(1-phenylethylidene) andrographolide; 3,19-O-Benzylidene-14-deoxy-12-(2, 3-dimethylanilino) andrographolide; 3,19-0-Benzylidene-14-deoxy-12α-(2, 3-dimethylanilino) andrographolide; 3,19-O-Benzylidene-14-deoxy-12β-(2,3-dimethylanilino) andrographolide; 3,19-Diacetyl-12-anilino-14-deoxy-8,17-epoxy andrographolide; 3,19-Diacetyl-12α-anilino-14-deoxy-8, 17-epoxy andrographolide; 3,19-Diacetyl-12β-anilino-14-deoxy-8, 17-epoxy andrographolide; 3,19-Diacetyl-14-deoxy-8, 17-epoxy-12-(2, 3-dimethylanilino) andrographolide; 3,19-Diacetyl-14-deoxy-8, 17-epoxy-12α-(2,3-dimethylanilino) andrographolide; 3,19-Diacetyl-14-deoxy-8, 17-epoxy-12β-(2, 3-dimethylanilino) andrographolide; 14-Deoxy-12-(N¹-uracil) andrographolide; 14-Deoxy-12α-(N¹-uracil) andrographolide; 14-Deoxy-12β-(N¹-uracil) andrographolide; 3,19-Diacetyl-14-deoxy-12-[N-(1, 2-dihydro-2-pyrimidinone) amino]-1-andrographolide; 3,19-Diacetyl-14-deoxy-12α-[N-(1,2-dihydro-2-pyrimidinone) amino]-1-andrographolide; 3, 19-Diacetyl-14-deoxy-12β-[N-(1, 2-dihydro-2-pyrimidinone) amino]-1-andrographolide; 3,19-Diacetyl-14-deoxy-12-(N¹-uracil) andrographolide; 3,19-Diacetyl-14-deoxy-12α-(N¹-uracil) andrographolide; 3,19-Diacetyl-14-deoxy-12β-(N¹-uracil) andrographolide; 3, 19-Diacetyl-1 4-deoxy-12-[N¹(5-chlorouracil)] andrographolide; 3, 19-Diacetyl-14-deoxy-12β-[N¹-(5-chlorouracil)]andrographolide; 3,19-Diacetyl-14-deoxy-12β-[N-(5-chlorouracil)] andrographolide; 3, 19-Diacetyl-14-deoxy-12-[N¹-(5-bromouracil)] andrographolide; 3,19-Diacetyl-1 4-deoxy-12α-[N¹-(5-bromouracil) Jandrographolide; 3,19-Diacetyl-14-deoxy-12β-[N¹-(5-bromouracil)] andrographolide; 3,19-Diacetyl-14-deoxy-12-[N¹-(5-fluorouracil] andrographolide; 3,19-Diacetyl-14-deoxy-12α-[N¹-(5-fluorouracil] andrographolide; 3,19-Diacetyl-1 4-deoxy-1 2β-[N-(5-fluorouracil)] andrographolide; 3, 19-Diacetyl-1 4-deoxy-12-[N¹-(5-iodouracil)] andrographolide; 3,19-Diacetyl-14-deoxy-12α-(5-iodouracil)] andrographolide; 3,19-Diacetyl-14-deoxy-12β-[N¹-(5-iodouracil)] andrographolide;; 14-Deoxy-12-[N-(1, 2-dihydro-2-pyrimidinone) amino] andrographolide; 14-Deoxy-12α-[N-(1, 2-dihydro-2-pyrimidinone) amino] andrographolide; 14-Deoxy-12β-[N-(1, 2-dihydro-2-pyrimidinone) amino] andrographolide; 14-Deoxy-12-[NI-(5-fluorouracil)] andrographolidep; 14-Deoxy-12α-[N-(5-fluorouracil)] andrographolide; 14-Deoxy-12β-[N¹-(5-fluorouracil)] andrographolide; 14-Deoxy-12-[N¹-(5-bromouracil)] andrographolide; 14-Deoxy-12α-[N¹-(5-bromouracil)] andrographolide; 14-Deoxy-12β-[N¹-(S-bromouracil)] andrographolide; 14-Deoxy-12-[N¹-(5-iodouracil)] andrographolide; 14-Deoxy-12α-[N¹-(5-iodouracil)] andrographolide; 14-Deoxy-12β-[N¹-(5-iodouracil)] andrographolide; 14-Deoxy-8,17-epoxy-12-phenylthio andrographolide; 14-Deoxy-8, 17-epoxy-12α-phenylthio andrographolide; 14-Deoxy-8, 17-epoxy-12β-phenylthio andrographolide; 3,19-Diacetyl-14-deoxy-12-phenylseleno andrographolide; 3,19-Diacetyl-14-deoxy-12α-phenylseleno andrographolide; 3,19-Diacetyl-14-deoxy-12β-phenylseleno andrographolide; 12-(C-Benzoylmethyl)-14-deoxy-13, 19-O-(1-phenylethylidene) andrographolide; 12α-(C-Benzoylmethyl)-14-deoxy-13, 19-O-(1-phenylethylidene) andrographolide; 12β-(C-Benzoylmethyl)-14-deoxy-13, 19-O-(1-phenylethylidene) andrographolide, 14-Deoxy-3,19-O-isopropylidene-12-ethylthio andrographolide; 14-Deoxy-3,19-O-isopropylidene-12α-ethylthio andrographolide; 14-Deoxy-3,19-0-isopropylidene-12β-ethylthio andrographolide; 3,19-Diacetyl-14-deoxy-12-phenylthio andrographolide; 3,19-Diacetyl-14-deoxy-12α-phenylthio andrographolide; 3,19-Diacetyl-14-deoxy-12β-phenylthio andrographolide; 3,19-Diacetyl-14-deoxy-12-acetylthio andrographolide; 3,19-Diacetyl-14-deoxy-12α-acetylthio andrographolide; 3,19-Diacetyl-14-deoxy-12β-acetylthio andrographolide; 3,19-Diacetyl-14-deoxy-12-ethylthio andrographolide; 3,19-Diacetyl-14-deoxy-12α-ethylthio andrographolide; 3,19-Diacetyl-14-deoxy-12β-ethylthio andrographolide; 3,19-Diacetyl-12-benzyl-14-deoxy andrographolide; 3,19-Diacetyl-12α-benzyl-14-deoxy andrographolide; 3,19-Diacetyl-12β-benzyl-14-deoxy andrographolide; 3,19-Diacetyl-14-deoxy-12-(1, 1′-diethyl dicarboxylate methyl) andrographolide; 3,1 9-Diacetyl-14-deoxy-12α-(1, 1′-diethyl dicarboxylate methyl) andrographolide; 3,19-Diacetyl-14-deoxy-12β-(1, 1′-diethyl dicarboxylate methyl) andrographolide; 14-Deoxy-12-phenylthio andrographolide; 14-Deoxy-12α-phenylthio andrographolide; 14-Deoxy-12β-phenylthio androgapholide; 14-Deoxy-12-ethylthio andrographolide; 14-Deoxy-12α-ethylthio andrographolide; 14-Deoxy-12β-ethylthio andrographolide; 14-Deoxy-12-phenylseleno andrographolide; 14-Deoxy-12α-phenylseleno andrographolide; 14-Deoxy-12β-phenylseleno andrographolide; 14-Deoxy-3, 19-O-isopropylidene-12-phenylthio andrographolide; 14-Deoxy-3,19-O-isopropylidene-12α-phenylthio andrographolide; 14-Deoxy-3,19-O-isopropylidene-12β--phenylthio andrographolide; 14-Deoxy-3,19-O-(1-phenylethylidene)-12-phenylthio andrographolide; 14-Deoxy-3,19-O-(1-phenylethylidene)-12α-phenylthio andrographolide; 14-Deoxy-3, 19-O-(1-phenylethylidene)-12β-phenylthio andrographolide; 14-Deoxy-3, 19-O-(1-phenylethylidene)-12-ethylthio andrographolide; 14-Deoxy-3,19-O-(1-phenylethylidene)-12α-ethylthio andrographolide; 14-Deoxy-3,19-O-(1-phenylethylidene)-12β-ethylthio andrographolide; 3,19-O-Benzylidene-14-deoxy-12-phenylthio andrographolide; 3, 19-O-Benzylidene-14-deoxy-12α-phenylthio andrographolide; 3,19-O-Benzylidene-14-deoxy-12β-phenylthio andrographolide; 3,19-Diacetyl-14-deoxy-8, 17-epoxy-12-phenylthio andrographolide; 3,19-Diacetyl-14-deoxy-8, 17-epoxy-12α-phenylthio andrographolide; 3,19-Diacetyl-14-deoxy-8, 17-epoxy-12β-phenylthio andrographolide; 12-Cimmamoyloxy-14-deoxy andrographolide; 12α-Cinnamoyloxy-14-deoxy andrographolide; 12β-Cinnamoyloxy-14-deoxy andrographolide; 12-Cinnamoyloxy-14-deoxy-8, 17-epoxy andrographolide; 12α-Cinnamoyloxy-14-deoxy-8, 17-epoxy andrographolide; 12β-Cinnamoyloxy-14-deoxy-8, 17-epoxy andrographolide; 14-Deoxy-12-hydroxy andrographolide; 14-Deoxy-12α-hydroxy andrographolide; 14-Deoxy-12β-hydroxy andrographolide; 12-Acetoxy-3,19-diacetyl-14-deoxy andrographolide; 12α-Acetoxy-3,19-diacetyl-14-deoxy andrographolide; 12β-Acetoxy-3, 19-diacetyl-14-deoxy andrographolide; 3,19-Diacetyl-14-deoxy-12-methoxy andrographolide; 3,19-Diacetyl-14-deoxy-12α-methoxy andrographolide; 3, 19-Diacetyl-14-deoxy-12β-methoxy andrographolide; 3,19-Diacetyl-14-deoxy-12-(2-acetoxy-3-N-acetylamino-3-phenylpropionyloxy) andrographolide; 3,19-Diacetyl-14-deoxy-12α-(2-acetoxy-3 -N-acetylamino-3-phenylpropionyloxy) andrographolide; 3,19-Diacetyl-14-deoxy-12β-(2-acetoxy-3-N-acetylamino-3-phenylpropionyloxy) andrographolide; 12-(N-Boc glycinyloxy)-14-deoxy-8, 17-epoxy-3, 19-dipropionyl andrographolide; 12α-(N-Boc glycinyloxy)-14-deoxy-8,17-epoxy-3,19-dipropionylandrographolide; 12β-(N-Boc glycinyloxy)-14-deoxy-8, 17-epoxy-3,19-dipropionyl andrographolide; 3,19-Diacetyl-14-deoxy-12-mercaptobenzothiazolyl andrographolide; 3,19-Diacetyl-14-deoxy-12α-mercaptobenzothiazolyl andrographolide; 3,19-Diacetyl-14-deoxy-12β-mercaptobenzothiazolyl andrographolide; 3,19-Diacetyl-12-(N, N-benzylchloroacetyl) amino-14-deoxy-12-andrographolide; 3,19-Diacetyl-12α-(N, N-benzylchloroacetyl) amino-14-deoxy-12-andrographolide and 3,19-Diacetyl-12β-(N, N-benzylchloroacetyl) amino-14-deoxy-12-andrographolide.

The compounds of the invention can be made by isolation from a plant Andrographis paniculata (Burm. f.) Nees, (Acanthaceae). Alternatively the compound may be synthesised using methods known in the art.

### Treatment Methods

“Treatment” and “treat” and synonyms thereof refer to both therapeutic treatment and prophylactic or preventative measures, wherein the object is to prevent or slow down (lessen) an airway disorder. An airway disorder may include asthma, an asthma exacerbation, chronic obstructive pulmonary disease (COPD) and other airway disorders known to those skilled in the art.

As used herein a “therapeutically effective amount” of a compound will be an amount of active agent that is capable of treating, preventing or at least slowing down (lessening) an airway disorder. Dosages and administration of an antagonist of the invention in a pharmaceutical composition may be determined by one of ordinary skill in the art of clinical pharmacology or pharmacokinetics. An effective amount of the compound or composition to be employed therapeutically will depend, for example, upon the therapeutic objectives, the route of administration, and the condition of the mammal. Accordingly, it will be necessary for the therapist to titer the dosage and modify the route of administration as required to obtain the optimal therapeutic effect. A typical daily dosage might range from about 10 ng/kg to up to 100 mg/kg of the mammal's body weight or more per day, preferably about 1 μmg/kg/day to 10 mg/kg/day.

“Subject” for the purposes of the present invention includes humans and other animals, particularly mammals. Thus the methods are applicable to both human therapy and veterinary applications. In certain embodiments the subject is a mammal, and in a preferred embodiment the subject is human.

“Therapeutically effective amount” is an amount of a compound of the invention, that when administered to a patient, ameliorates a symptom of the disease. The amount of a compound of the invention which constitutes a “therapeutically effective amount” will vary depending on the compound, the disease state and its severity, the age and weight of the patient to be treated, and the like. The therapeutically effective amount can be determined routinely by one of ordinary skill in the art having regard to their knowledge and to this disclosure.

### Compositions of the Invention

Compounds produced according to the invention can be administered for the treatment of airway disorders in the form of pharmaceutical compositions.

Thus, the present invention also relates to compositions including pharmaceutical compositions comprising a therapeutically effective amount of a compound of the invention. As used herein a compound will be therapeutically effective if it is able to affect the measured parameters of airway inflammation.

In a preferred embodiment the compounds and compositions are adapted to be administered to the lungs directly through the airways by inhalation. Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose or starch. Inhalable dry powder compositions may be presented in capsules and cartridges of gelatin or a like material, or blisters of laminated aluminium foil for use in an inhaler or insufflator. Each capsule or cartridge may generally contain between 20 pg-10 mg of the active compound. Alternatively, the compound of the invention may be presented without excipients.

The inhalable compositions may be packaged for unit dose or multi-dose delivery. For example, the compositions can be packaged for multi-dose delivery in a manner analogous to that described in GB 2242134, U.S. Pat. No. 6,632,666, U.S. Pat. No. 5,860,419, U.S. Pat. No. 5,873,360 and U.S. Pat. No. 5,590,645 (all illustrating the “Diskus” device), or GB2178965, GB2129691, GB2169265, U.S. Pat. No. 4,778,054, U.S. Pat. No. 4,811,731 and U.S. Pat. No. 5,035,237 (which illustrate the “Diskhaler” device), or EP 69715 (“Turbuhaler” device), or GB 2064336 and U.S. Pat. No. 4,353,556 (“Rotahaler” device).

Spray compositions for topical delivery to the lung by inhalation may be formulated as aqueous solutions or suspensions or as aerosols delivered from pressurised packs, such as a metered dose inhaler (MDI), with the use of a suitable liquefied propellant. The medication in pressurized MDI is most commonly stored in solution in a pressurized canister that contains a propellant, although it may also be a suspension.

Aerosol compositions suitable for inhalation can be presented either as suspensions or as solutions and typically contain the active compound and a suitable propellant such as a fluorocarbon or hydrogen-containing chlorofluorocarbon or mixtures thereof, particularly hydrofluoroalkanes such as dichlorodifluoromethane, trichlorofluoromethane, dichlorotetrafluoroethane, and especially 1,1, 1, 2-tetrafluoroethane, 1,1, 1,2, 3,3, 3-heptafluoro-n-propane and mixtures thereof

The aerosol composition may optionally contain additional excipients typically associated with such compositions, for example surfactants such as oleic acid or lecithin and cosolvents such as ethanol. Pressurised formulations will generally be contained within a canister (for example an aluminium canister) closed with a metering valve and fitted into an actuator provided with a mouthpiece.

Medicaments for administration by inhalation desirably have a controlled particle size. The optimum particle size for inhalation into the bronchial system is usually 1-10 μm, preferably 2-5 μm. Particles having a size above 20 μm are generally too large when inhaled to reach the small airways. To achieve these particle sizes the particles of the active ingredient may be subjected to a size reducing process such as micronisation. The desired size fraction may be separated out by air classification or sieving. Preferably, the particles will be crystalline. When an excipient such as lactose is employed, typically the particle size of the excipient will be much greater than the particle size of the active ingredient.

Intranasal sprays may be formulated with aqueous or non-aqueous vehicles with the addition of agents such as thickening agents, buffer salts or acid or alkali to adjust the pH, isotonic adjusting agents or anti-oxidants.

Solutions for inhalation by nebulisation may be formulated with an aqueous vehicle with the addition of agents such as acid or alkali, buffer salts, isotonic adjusting agents or antimicrobial agents. They may be sterilised by filtration or heating in an autoclave, or presented as a non-sterile product. Nebulizers supply the aerosol as a mist created from an aqueous formulation.

In one particular embodiment the composition is administered from a dry powder inhaler.

In another embodiment, the composition is administered by an aerosol dispensing device, preferably in conjunction with an inhalation chamber such as the “Volumatic” (RTM) inhalation chamber.

Pharmaceutical forms of the invention suitable for injectable use include sterile aqueous solutions (where water soluble) or dispersions and sterile powders for the extemporaneous preparation of sterile injectable solutions and or one or more carrier. Alternatively, injectable solutions may be delivered encapsulated in liposomes to assist their transport across cell membrane. The composition must be stable under the conditions of manufacture and storage and must be preserved against the contaminating/destructive action of microorganisms such as, for example, bacteria and fungi.

The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as, for example, lecithin, by the maintenance of the required particle size in the case of dispersion and by the use of surfactants. Preventing the action of microorganisms in the compositions of the invention is achieved by adding antibacterial and/or antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile inhalable or injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile inhalable or injectable solutions, the preferred methods of preparation are vacuum drying and freeze-drying, to yield a powder of the active ingredient plus any additional desired ingredient from previously sterile-filtered solution thereof.

When the active ingredients, in particular small molecules contemplated within the scope of the invention, are suitably protected they may be orally administered, for example, with an inert diluent or with an edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that a dosage unit form contains between about 0.1 μg and 20 g of active compound.

The tablets, troches, pills, capsules and the like may also contain binding agents, such as, for example, gum, acacia, corn starch or gelatin. They may also contain an excipient, such as, for example, dicalcium phosphate. They may also contain a disintegrating agent such as, for example, corn starch, potato starch, alginic acid and the like. They may also contain a lubricant such as, for example, magnesium stearate. They may also contain a sweetening agent such a sucrose, lactose or saccharin. They may also contain a flavouring agent such as, for example, peppermint, oil of wintergreen, or cherry flavouring.

When the dosage unit form is a capsule, it may contain, in addition to materials of the above type, a liquid carrier.

Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparaben as preservatives, a dye and flavouring such as, for example, cherry or orange flavour. Of course, any material used in preparing any dosage unit form should be pharmaceutically pure and substantially non-toxic in the amounts employed. In addition, the active compound(s) may be incorporated into sustained-release preparations and formulations.

To this extent the active ingredient may be held within a matrix which controls the release of the active agent. Preferably, the matrix comprises a substance selected from the group consisting of lipid, polyvinyl alcohol, polyvinyl acetate, polycaprolactone, poly(glycolic)acid, poly(lactic)acid, polycaprolactone, polylactic acid, polyanhydrides, polylactide-co-glycolides, polyamino acids, polyethylene oxide, acrylic terminated polyethylene oxide, polyamides, polyethylenes, polyacrylonitriles, polyphosphazenes, poly(ortho esters), sucrose acetate isobutyrate (SAIB), and combinations thereof and other polymers such as those disclosed in U.S. Pat. Nos. 6,667,371; 6,613,355; 6,596,296; 6,413,536; 5,968,543; 4,079,038; 4,093,709; 4,131,648; 4,138,344; 4,180,646; 4,304,767; 4,946,931, each of which is expressly incorporated by reference herein in its entirety. Preferably, the matrix sustainedly releases the drug.

Pharmaceutically acceptable carriers and/or diluents may also include any and all solvents, dispersion media, coatings, antibacterials and/or antifungals, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated.

Supplementary active ingredients can also be incorporated into the compositions. Preferably those supplementary active ingredients are anti-inflammatory agents such as inhaled steroids, cysteinyl-leukotriene receptor antagonist and cromolyn and or bronchodilators such as (3₂ agonists and/or anticholinergics. Some inhaled steroids may include Dexamethasone, Budesonide (Pulmicort®), Fluticasone (Flovent®), Ciclesonide (Alvesco®), Beclomethasone Dipropionate (QVAR®) or others known in the art. β₂ agonists may include salbutamol, albuterol, terbutaline, salmeterol, or formoterol. An anticholinergic may include Ipratropium.

It is especially advantageous to formulate parenteral compositions in dosage unit form for ease of administration and uniformity of dosage. Dosage unit form as used herein refers to physically discrete units suited as unitary dosages for the mammalian subjects to be treated, each unit containing a predetermined quantity of active material calculated to produce the desired therapeutic effect in association with the required pharmaceutical carrier. The dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired as herein disclosed in detail.

The principal active ingredient is compounded for convenient and effective administration in effective amounts with a suitable pharmaceutically acceptable carrier in dosage unit form. A unit dosage form can, for example, contain the principal active compound in amounts ranging from 0.5 μg to about 2000 mg. Expressed in proportions, the active compound is generally present in from about 0.5 pg to about 2000 mg/ml of carrier. In the case of compositions containing supplementary active ingredients, the dosages are determined by reference to the usual dose and manner of administration of-the said ingredients.

The compound or the composition may be in the form of a treatment kit comprising the dosage unit forms and instructions for use.

Preferably Andrographolide or 14-deoxy-1 1,12-didehydroandrographolide (DDAG) may be able to replace or reduce the dosage of glucocorticoid either with DDAG alone or by combination therapy of DDAG and glucocorticoid in the treatment of asthma.

### Preferred Embodiments

We propose the use of andrographolide compounds such as andrographolide and DDA (DDAG) to complement or to replace oral steroids during airway disorders such as asthma exacerbation.

We describe a Novel Anti-Inflammatory Role for Andrographolide in Asthma via Inhibition of The Nuclear Factor-κB Pathway. Persistent activation of nuclear factor (NF)-κB has been associated with the development of asthma. Andrographolide, the principal active component of a medicinal plant Andrographis paniculata, has been shown here to inhibit NF-κB activity. Andrographolide attenuates allergic asthma via inhibition of the NF-κB signaling pathway in BALB/c mice sensitized and challenged with OVA developed airway inflammation.

Andrographolide inhibited OVA-induced increases in total cell count, eosinophil count, and IL-4, IL-5 and IL-13 levels in bronchoalveolar lavage fluid, and reduced serum level of OVA-specific IgE. It attenuated OVA-induced lung tissue eosinophilia and airway mucus production, mRNA expression of E-selectin, chitinases, MucSac and inducible nitric oxide synthase in lung tissues, and airway hyperresponsiveness to methacholine. In human lung epithelial cells, andrographolide blocked TNF-α-induced phosphorylation of inhibitory κB (IκB) kinase-β (IKKβ), and downstream IκBαdegradation, p65 subunit of NF-κB phosphorylation, and p65 nuclear translocation and DNA-binding activity. Our findings implicate a potential therapeutic value of andrographolide in the treatment of asthma and it may act by inhibiting NF-κB pathway at the level of IKKβ activation.

We describe for the first time that DDAG effectively reduced OVA-induced inflammatory cell recruitment into BAL fluid, IL-4, IL-5, IL-13 and eotaxin production, serum IgE synthesis, pulmonary eosinophilia, mucus hypersecretion and AHR in a mouse asthma model potentially via inhibition of NF-κB activity. Moreover, at doses that do not show any anti-inflammatory effects when given alone, combination of low dose of DDAG and glucocorticoid treatment synergistically attenuated inflammation in mouse asthma model. These findings support a therapeutic value for DDAG in the treatment of asthma.

We propose to use andrographolide or 14-deoxy-11,12-didehydroandrograholide (DDA) for the treatment of asthma as a controller. In addition, 5-10% of the asthmatics are not well-controlled by current drug treatment and they require oral steroids during exacerbation. We propose to use andrographolide or DDA to complement oral steroids or replace oral steroid during asthma exacerbation.

### Use of Andrographolide in Treating Airway Disorders

A novel anti-Inflammatory role of andrographolide in treating asthma via Inhibition of the NF-κB pathway. In our mouse asthma model using ovalbumin as aeroallergen, we showed that andrographolide dose-dependently Inhibited ovalbumin-Induced cell Infiltration Into the airways obtained from bronchoalveolar lavage fluid and observed in formalin-fixed lungs as shown in FIG. 2

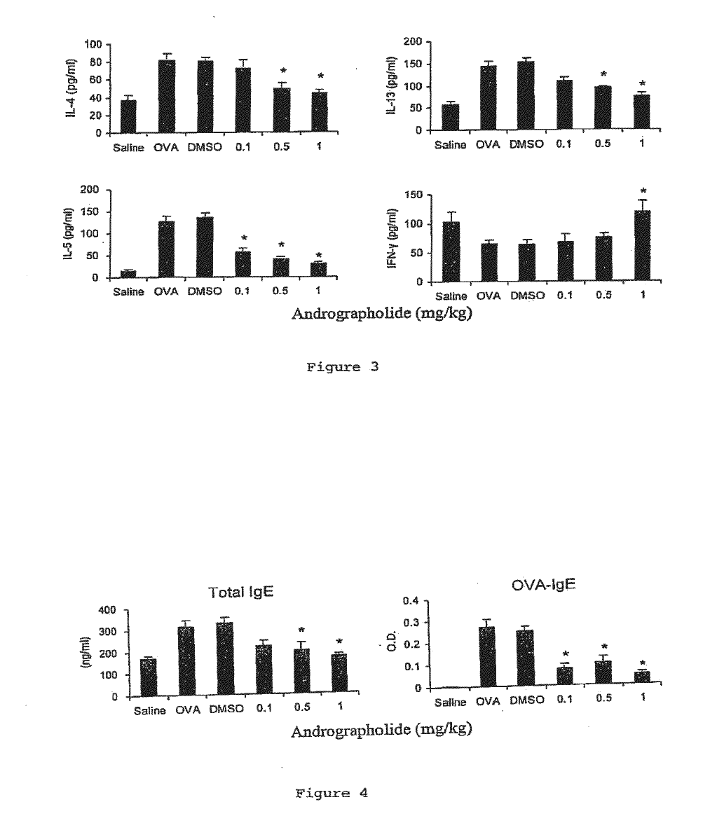

Andrographolide was able to suppress ovalbumln-Induced cytoklne production obtained from BAL fluid (FIG. 3) and serum IgE levels (FIG. 4). This indicates that andrographolide may have anti-allergy and anti-inflammatory activity.

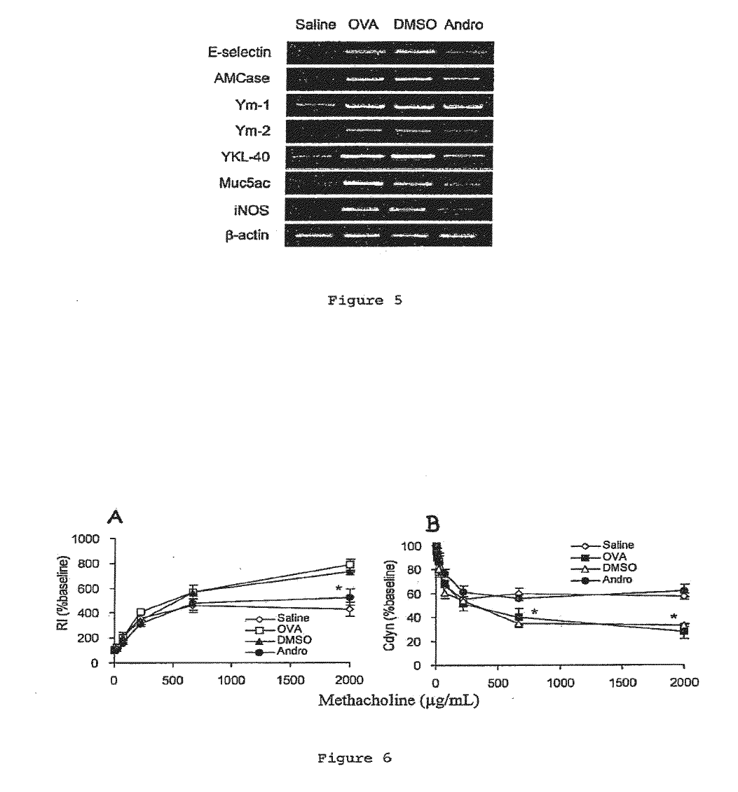

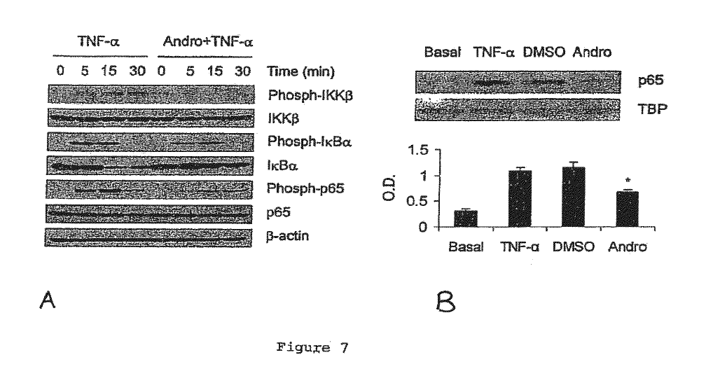

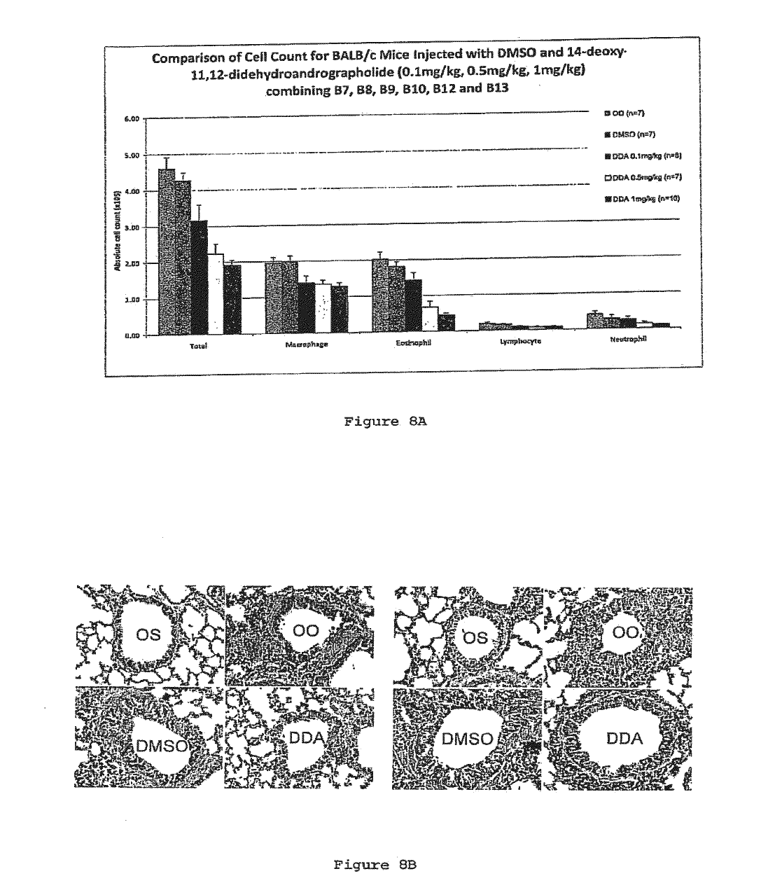

Furthermore, andrographolide was able to suppress ovalbumin-induced expression pro-inflammatory adhesion molecules and biomarkers (FIG. 5). The clinical endpoint of airway hyperresponslveness in mice could also be blocked by andrographolide (FIG. 6). The mechanism of anti-inflammatory actions of andrograhpolide in asthma is likely associated with Its Inhibitory effect on nuclear factor-κB signalling pathway (FIG. 7). Protein bands of IKKβ and phospho-IKKβ were quantltated using Gel-Pro Imaging software (Media Cybernetics, Silver Spring, Md.). β-actin was used as an Internal control. (Upper right) Immunoblotting of p65 level in nuclear extracts of epithelial cells stimulated with TNF-α for 30 minutes in the presence and absence of 30 μM andrographolide. Nuclear proteins were separated by 10% SDS-PAGE and probed with anti-p65 or anti-TBP antibody. TBP nuclear protein was used as an internal control. (Lower right) DNA-binding activity of p65 NF-κB In nuclear extracts of epithelial cells stimulated with TNF-α for 30 minutes in the presence and absence of 30 μM andrographolide was determined using a TransAM™ p65 transcription factor ELISA kit.

Andrgrapholide reduced ovalbumin-induced eosinophil count in BAL fluid and it inhibited E-selectin and VCAM-1 staining in lung sections (FIGS. 2 to 7). Andrograpoholide inhibited OVA-induced eosinophil and lymphocyte counts and TNF-α and GM-CSF levels in BAL fluid. Our findings revealed several anti-asthma properties of andrographolide, which include inhibition of cell inflammation, infiltration into the airways using H & E staining, mucus secretion in the airways using PAS staining, Th2 cytoklne (IL-4, IL-5, IL-13 and eotaxin) level in BAL fluid, serum IgE level (total IgE, OVA-specific IgE, IgG1 and IgG2a), airway hyper-responsiveness as reflected by airway resistance and dynamic compliance, and proinflammatory marker gene expression Including ICAM-1, VCAM-1, E-selectin, AMCase, YKL-40, YM1, YM2, MUC5ac and inOS. We have also reported a new mechanism of action of andrographolide by showing the inhibition of IKKβ phosphorylatlon, IκB phosphorylatlon, IκBα degradation, p65 nuclear translocation and p65-DNA binding. We have provided a detailed picture of how andrographolide works in asthma. We also showed potential anti-allergy action of andrographolide.

### Use of DDAG in Treating Airway Disorders

In addition, we have data showing DDAG (14-deoxy-1 1,12-didehydroandrographolide) anti-inflammatory effects in asthma.

DDAG dose-dependently inhibited ovalbumin-induced increases in total cell count, eosinophil count, and IL-4, IL-5 and IL-13 levels recovered in bronchoalveolar lavage fluid (BALF), and reduced serum level of ovalbumin-specific IgE. It attenuated ovalbumin-induced lung tissue eosinophilia and airway mucus production, mRNA expression of E-selectin, chitinases, COX-2, IL-17, IL-33 and Muc5ac in lung tissues, and airway hyper-responsiveness to methacholine. In normal human bronchial epithelial cells, DDAG blocked TNF-α-induced p65 nuclear translocation and DNA binding activity. Similarly, DDAG blocked p65 nuclear translocation and DNA-binding activity in the nuclear extracts from lung tissues of ovalbumin-challenged mice.

Additionally, low dose Dexamathasone combination synergistically inhibited BALF ovalbumin-induced total cell count, eosinophil count and IL-4, IL-5, IL-13 and even Eotaxin levels.

In the mouse asthma model using ovalbumin as aeroallergen, we showed that DDA dose-dependently inhibited ovalbumin-induced cell Infiltration into the airways obtained from bronchoalveolar lavage fluid FIG. 8A and observed in formalin-fixed lungs as shown in FIG. 8B.

Several Inflammatory marker gene expression profiles are inhibited by DDA and the Inhibitory effects of DDA on airway hyper-responsiveness.

We propose the use of andrographolide and DDA as an anti-inflammatory agent for controlling asthma.

BALB/c mice sensitized and challenged with ovalbumin developed airway inflammation. Bronchoalveolar lavage fluid was assessed for total and differential cell counts, and cytokine and chemokine levels. Serum IgE levels were also determined. Lung tissues were examined for cell infiltration and mucus hypersecretion, and the expression of inflammatory biomarkers. Airway hyperresponsiveness was monitored by direct airway resistance analysis.

DDAG elicited a significant inhibition on p65 nuclear translocation and κB DNA-binding activity in OVA-challenged lungs in vivo and in normal human bronchial epithelial cells in vitro. Reduction in IL-4, IL-5, IL-13 and eotaxin in BAL fluids may be due to inhibition of NF-κB by DDAG in inflammatory and airway resident cells. Reduction in airway eosinophilia by DDAG may be due to decreased IL-13, eotaxin, RANTES and E-selectin expression, secondary to NF-κB inhibition.

Reduction in serum total and OVA-specific IgE by DDAG may be due to inhibitory effect on B cell activation via inhibition of NF-κB. Therefore, reduction in airway hyper-responsivness by DDAG was observed. Although DDAG is not as active as steroid, it demonstrate significant synergistic anti inflammatory effect when a low dose of DDA was use in combination with low dose of steroid.

### DDAG Suppresses OVA-Induced Inflammatory Cell Recruitment and Mucus Production

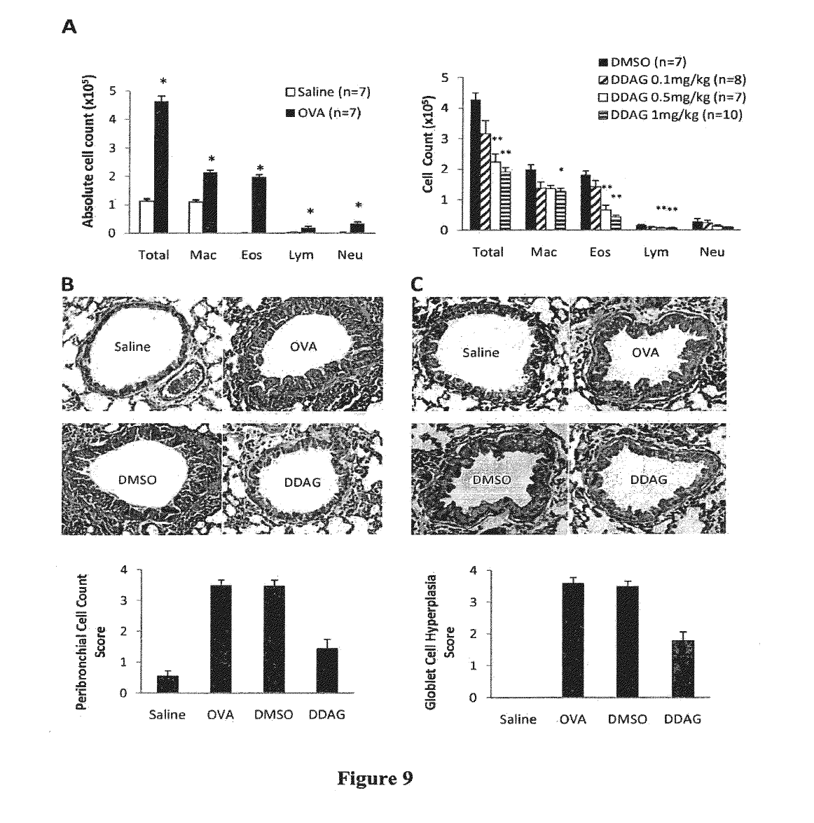

BAL fluid was collected 24 hours after the last OVA or saline aerosol challenge, and total and differential cell counts were performed. OVA inhalation markedly increased total cell and eosinophil counts, but slightly yet significantly (P<0.05) increased macrophage, lymphocyte and neutrophil counts, as compared with saline aerosol control. DDAG (0.1, 0.5 and 1 mg/kg) drastically decreased the total cell and eosinophil counts in BAL fluid in a dose-dependent manner as compared with the DMSO vehicle control (FIG. 9A). At high dose (1 mg/kg), DDAG also reduced macrophage and lymphocyte counts.

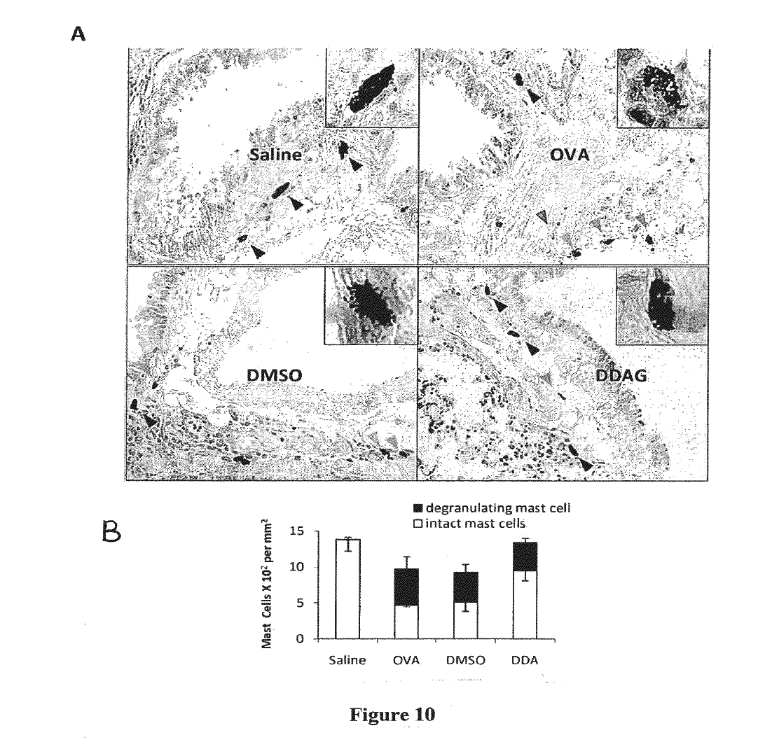

Lung tissue was also collected 24 hours after the last OVA or saline aerosol challenge. OVA aerosol challenge induced marked infiltration of inflammatory cells into the peribronchiolar and perivascular connective tissues as compared with saline aerosol challenge. DDAG (1 mg/kg) markedly diminished the eosinophil-rich leukocyte infiltration as compared with DMSO control (FIG. 9B). On the other hand, OVA-challenged mice, but not saline-challenged mice, developed marked goblet cell hyperplasia and mucus hypersecretion in the bronchi. OVA-induced mucus hypersecretion was significantly halted by DDAG (1 mg/kg) (FIG. 9C). DDAG was shown to reduce the number of degranulated mast cells in the lung tissue of OVA challenged mice (FIG. 10).

### DDAG Reduces OVA-Induced BAL Fluid Th2 Cytokine Levels and Serum Ig Production

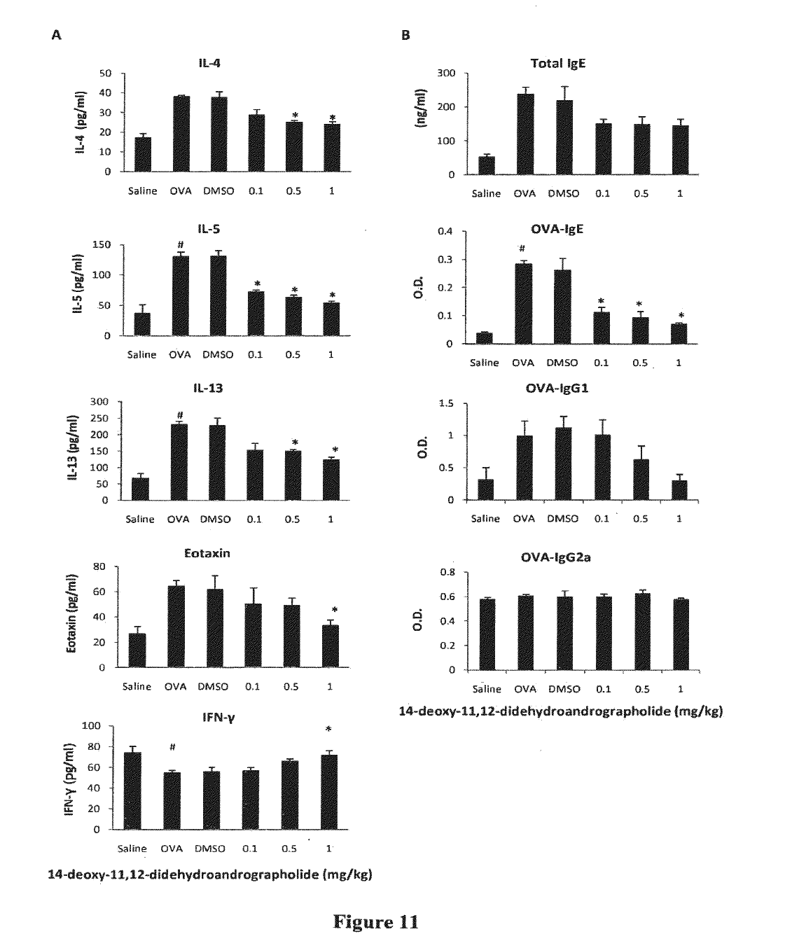

OVA inhalation in sensitized mice caused a notable increase in IL-4, IL-5, IL-13 and eotaxin levels into BAL fluid as compared with saline aerosol control (FIG. 11A). In contrast, BAL fluid level of IFN-γ, a Th1 cytokine, dropped slightly in OVA-challenged mice. DDAG significantly (P<0.05) reduced IL-4, IL-5 and IL-13, and to a lesser extent, eotaxin levels in BAL fluid in a dose-dependent manner as compared with DMSO control (FIG. 11A). Noticeably, DDAG at 1 mg/kg markedly upregulated IFN-γ level in BAL fluid. This finding implies that DDAG is able to modify the Th2-predominant immune activity in our OVA-induced mouse asthma model.

To further evaluate whether DDAG could modify an ongoing OVA-specific Th2 response in vivo, serum levels of total IgE, and OVA-specific IgE, IgG1 and IgG2a were determined using ELISA. Marked elevation in serum total IgE, OVA-specific IgE and OVA-specific IgG1 levels, but not OVA-specific IgG2a level, were observed in OVA-challenged mice as compared with saline-challenged mice (FIG. 11B). DDAG strongly suppressed OVA-specific IgE levels even at the lowest dose (0.1 mg/kg), and, to a lesser extent, the serum level of total IgE and OVA-specific IgG1 with significant effects at higher doses (FIG. 11B). DDAG had no effects on the serum level of OVA-specific IgG2a, indicating a specific inhibition of the Th2 response by DDAG.

### DDAG Reduces OVA-Induced AHR in Mice

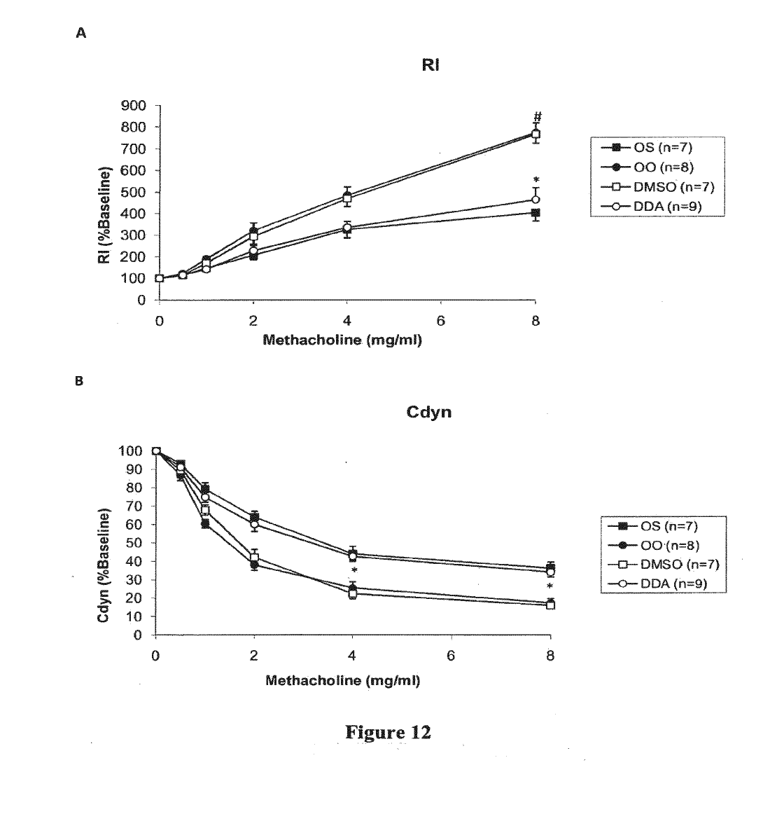

To investigate the effect of DDAG on AHR in response to increasing concentrations of methacholine, we measured both RI and Cdyn in mechanically ventilated mice. RI is defined as the pressure driving respiration divided by flow. Cdyn refers to the distensibility of the lung and is defined as the change in volume of the lung produced by a change in pressure across the lung. OVA-challenged mice developed AHR which is typically reflected by high RI and low Cdyn (FIG. 12). DDAG (1 mg/kg) dramatically reduced RI and restored Cdyn in OVA-challenged mice in response to methacholine, suggesting that immune-mediated airway pathology in vivo was modified. OVA inhalation markedly increased total degranulating mast cell counts as compared with saline aerosol control. DDAG (1 mg/kg) drastically decreased the total degranulating mast cell counts in toluidine-blue staining lung section as compared with the DMSO vehicle control (FIG. 12C).

DDAG Inhibits OVA-Induced Inflammatory Gene Expression and NO production in Allergic Airway Inflammation

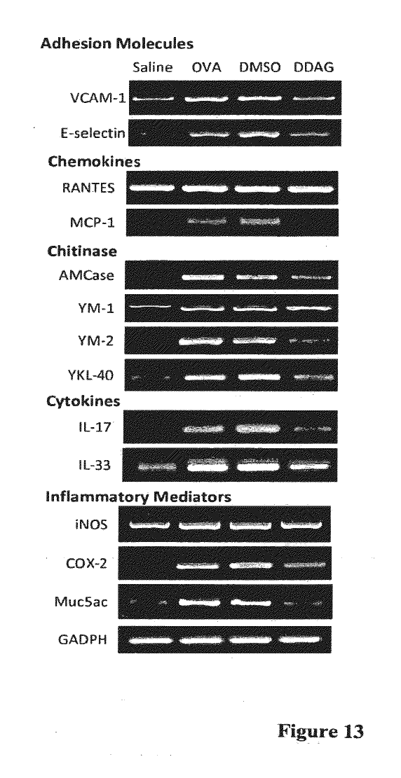

OVA aerosol challenge markedly up-regulated lung mRNA levels of adhesion molecule E-selectin, which is pivotal for pulmonary recruitment of inflammatory cells like eosinophils and lymphoctyes; chitinase family members including acidic mammalian chitinase (AMCase), Ym1, Ym2 and YKL-40, which have recently been shown to play critical roles in airway inflammation and remodeling²⁶⁻²⁸; Muc5ac, which is essential for mucus hypersecretion²⁹. IL-33 is known to recruit, activate and enhance Th2 T cell function³⁰; IL-17 has been shown to induce the release of eotaxin from airway smooth muscle cells, and both IL-17 and IL-17F were able to induce the release of in flammatory mediators from human eosinophils in vitro³¹. Pretreatment with DDAG (1 mg/kg) demonstrated strong suppression of E-selectin, AMCase, Ym-2, YKL-40, Muc5ac, COX2, IL-17 and IL-33 in the allergic airways (FIG. 13A). Nitric Oxide (NO) is produce in high concentration in response to inflammatory stimuli and perpetuate the inflammatory response within the airways. DDAG also reduce the serum nitrate nitrite level (FIG. 13B) which is the final product of NO in vivo.

DDAG Inhibits OVA-Induced NF-KB Function in Allergic Airway Inflammation and TNF-α-induced NF-κB Activation in Normal Human Bronchial Epithelial Cells

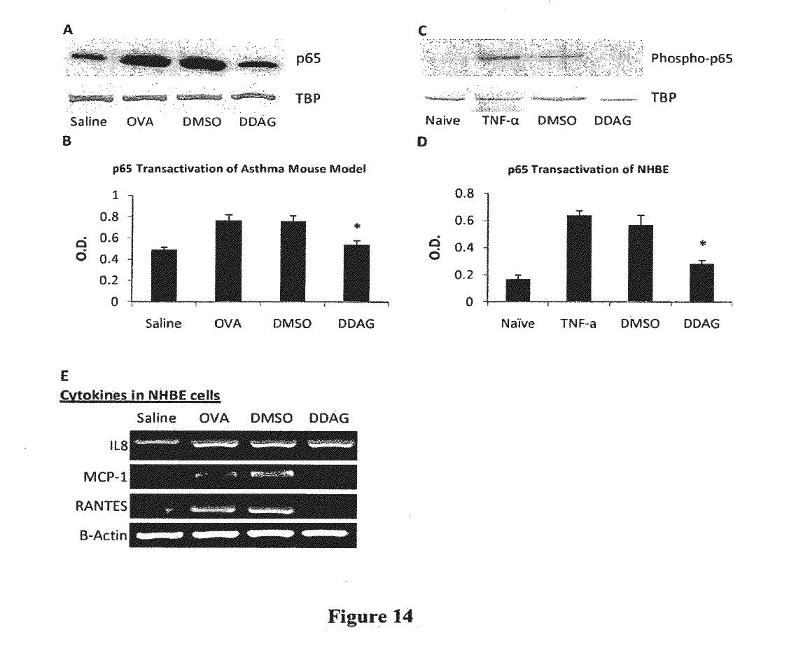

To verify that the anti-inflammatory effects of DDAG in OVA-challenged mice were mediated by the inhibition of NF-KB, we examined nuclear translocation of p65 subunit of NF-κB and p65 DNA-binding activity in lung tissues obtained 24 hours after the last OVA or saline aerosol challenge. OVA challenge markedly raised the level of p65 subunit in the nuclear extract of the lung tissue and promoted nuclear p65 DNA-binding activity as compared with saline aerosol control (FIGS. 14A and 14B). DDAG (1 mg/kg) significantly (P<0.05) reduced both nuclear p65 amount and DNA-binding activity to the basal levels, suggesting that DDAG may exert its anti-inflammatory actions via inhibition of NF-κB activity.

To further explore anti-inflammatory mechanisms of action of DDAG in a relevant airway cell type, we studied the effects of DDAG on TNF-α-induced activation of NF-κB and cytokine mRNA expression in normal primary human bronchial epithelial cells. TNF-α plays a critical role in asthma³²⁻³³ and is a potent stimulator of human airway epithelial cells³⁴. A sharp increase in nuclear p65 level and in p65 DNA-binding activity was observed (FIGS. 14C and 14D). DDAG markedly abated p65 nuclear translocation and DNA-binding (FIGS. 14A-14D). Furthermore, andrographolide noticeably blocked TNF-α-induced up-regulation of IL-6, IL-8 and RANTES mRNA expression in normal human bronchial epithelial cells (FIG. 14E).

Low dose of DDAG and Dexamethasone Synergistically Suppress OVA Induced Inflammatory Cell Recruitment and Reduces OVA-Induced BAL Fluid Th2 Cytokine Levels

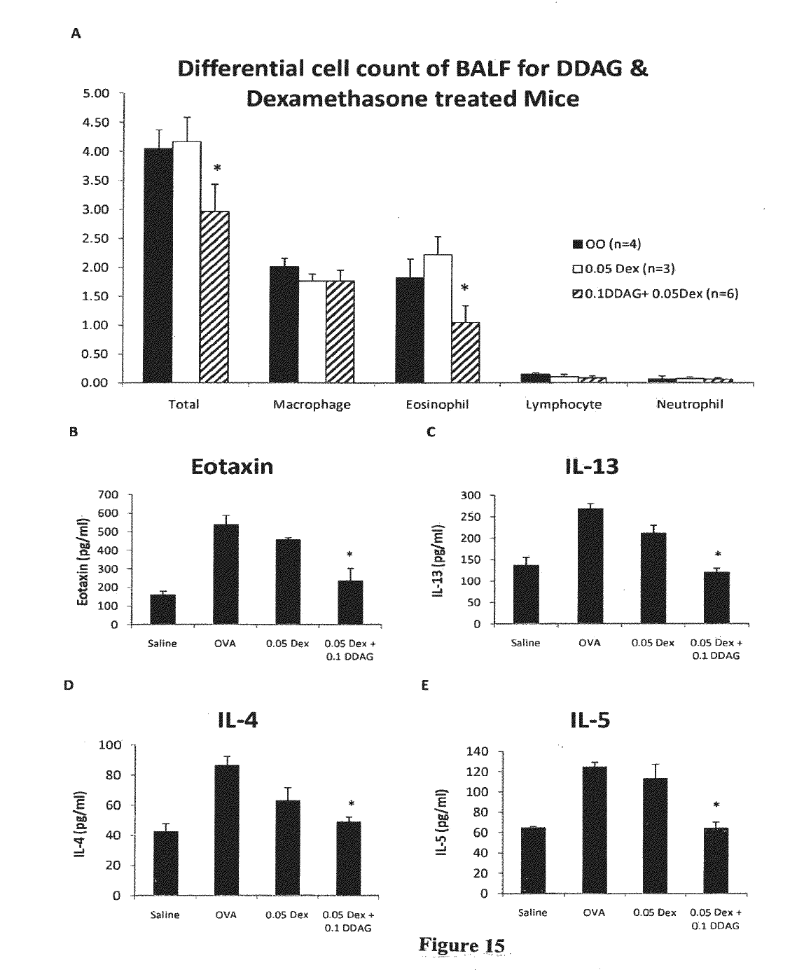

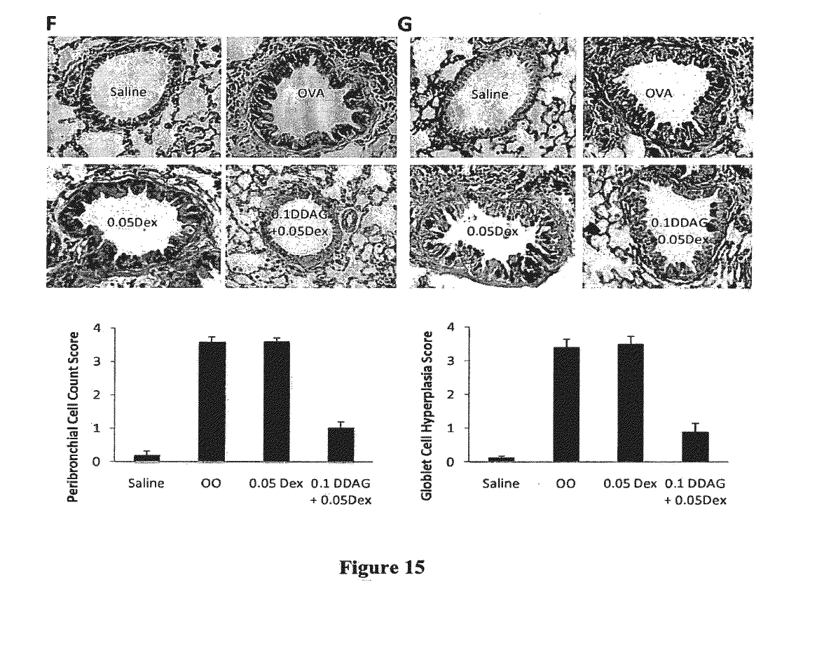

To study the combinational effect of DDAG and Glucocorticoids in OVA-induced airway inflammation, BAL fluid was collected 24 hours after the last OVA or saline aerosol challenge, and total and differential cell counts were performed. The lowest dose of DDAG (0.1 mg/kg) in combination with low dose of Dexamethasone (0.05 mg/kg) drastically decreased the total cell and eosinophil counts in BAL fluid as compared with the OVA sensitize and challenge but treated mice as positive control (FIG. 15A). DDAG in combination with low dose of steroids significantly (P<0.05) reduced IL-4, IL-5 and IL-13, and even eotaxin levels in BAL fluid in a dose dependent manner as compared with Dexamethasone only as control (FIG. 15 B to E).

### Discussion

Persistent NF-κB activation has been observed in allergic airway inflammation both in human and in animal models of asthma³⁵⁻³⁸. Antigen receptor activation in T and B lymphocytes and mast cells culminates in NF-κB activation³⁹⁻⁴⁰. In addition, TNF-α stimulation of airway epithelial cells triggers NF-icB-dependent gene expression³⁴. Various therapeutic strategies targeted at the NF-κB signaling pathway such as NF-κB-specific decoy oligonucleotide⁴¹, p65-specific antisense oligonucleotide⁴² and IMO-selective small molecule inhibitor⁴³ have demonstrated beneficial effects in experimental asthma models.

Our findings reveal a significant inhibition of p65 nuclear translocation and κB DNA binding activity by DDAG in OVA-challenged lungs in vivo. To be more specific, our immunoblotting analysis of TNF-α-stimulated normal human bronchial epithelial cells in vitro shows that andrographolide reduced nuclear translocation of p65, and diminished p65 κB oligonucleotide binding. Taken together, we have established that Andrographolide and DDAG, the principal active component of a medicinal plant Andrographis paniculata, can effectively suppress various aspects of OVA-induced Th2-mediated allergic airway inflammation in mice potentially via inhibition of NF-κB activity. As these compounds share the same basic structure as formula I (see FIGS. A and B) presumably other compounds containing the same basic structure as formula I will have a similar effect on airway inflammation.

Th2 cytokines play an essential role in the pathogenesis of the allergic airway inflammation¹⁻², and NF-κB is a critical transcription factor for Th2 cell differentiation⁴⁴. IL-4, IL-5 and IL-13 can be produced by various lung resident cells such as bronchial epithelial cells, tissue mast cells and alveolar macrophages as well as infiltrated inflammatory cells such as lymphocytes and eosinophils.

Our present results show that DDAG significantly reduced the levels of IL-4, IL-5, IL-13 and eotaxin in BAL fluids from OVA-challenged mice. Similar findings were observed in OVA-challenged mice with disrupted NF-κB function via conditional knockout of IKKβ or transgenic IκBα mutant expression selectively in airway epithelium^{38,45}. Consistently, expression of IL-33 which enhances the production of IL-5 and IL-13 by Th2 cells but not by Thl cells in vitro is drastically reduce by DDAG. In addition, repression of NF-κB signaling pathway has been shown to block IL-13-induced eotaxin production in cultured human airway smooth muscle cells⁴³. Therefore, the observed reduction of IL-4, IL-5, IL-13 and eotaxin levels in BAL fluid from DDAG-treated mice may be due to inhibition of NF-κB activation in the inflammatory and airway resident cells. These data show that the anti-inflammatory effect of DDAG is at least in part mediated through a suppressive action on T lymphocytes.

Eosinophils play a central role in the pathogenesis of allergic inflammation ^{5,7}. Our present findings showed that DDAG prevented inflammatory cell infiltration into the airways as shown by a significant drop in total cell counts and eosinophil and lymphocyte counts in BAL fluid, and in tissue eosinophilia in lung sections. Leukocyte transmigration into the airways is orchestrated by cytokines like IL-4, IL-5 and IL-13, and coordinated by specific chemokines like eotaxin and RANTES in combination with adhesion molecules such as VCAM-1 and E-selectin^{7,25}. IL-13 is by far the most potent inducer of eotaxin expression in airway epithelial cells⁴⁷. IL-17 has also been shown to induce Eotaxin from airway smooth muscle cell⁴⁸. We have demonstrated that DDAG strongly suppressed E-selectin and IL-17 mRNA expression and eotaxin production in OVA-challenged lungs, and RANTES mRNA expression in TNF-α-stimulated normal human bronchial epithelial cells. These findings are likely to be due to DDAG-mediated NF-κB inhibition, as the genes for Eselectin, eotaxin and RANTES contain the κB site for NF-κB within their promoters⁴⁹.

Taken together, the observed reduction in airway eosinophilia by andrographolide and DDAG may be a result of combined inhibitory effects on IL-13, eotaxin and RANTES production, and on E-selectin expression, secondary to inhibition of NF-κB activation. We have also demonstrated a dramatic reduction in airway mucus production in DDAG-treated mice as compared with DMSO control. Cumulative evidence indicates that IL-4, IL-5 and IL-13 play a critical role in goblet cell hyperplasia and mucin Muc5ac gene and protein expression in mice^{29,50}. Interestingly, Muc5ac gene expression is dependent on the transcriptional activity of NF-κB^{29, 49, 51}. We also observed a substantial drop in Muc5ac mRNA expression by andrographolide and DDAG in OVA-challenged lungs. Selective ablation of NF-κB function in airway epithelium has been shown to reduce OVA-induced mucus production in mice^{38, 45}. As such, the marked decrease in mucus production in the lungs of Eotaxin-treated mice may be attributable to a significant reduction of IL-4, IL-5 and IL-13 levels, and a direct inhibitory action on NF-κB in airway epithelium.

Elevated serum IgE levels are a hallmark of the Th2 immune response. Our data showed that serum levels of total IgE and OVA-specific IgE were substantially reduced by andrographolide and DDAG in OVA-challenged mice. In addition, NF-κB plays a crucial role in B cell proliferation and development^{39,52}, and IL-4 and IL-13 are important in directing B cell growth, differentiation and secretion of IgE^{3,6}. The biological activities of IgE are mediated through its interaction with the FcεRI on mast cells and basophils. Cross-linking of FcεRI initiates multiple signaling cascades leading to NF-κB activation and production of lipid mediators, cytokines and chemokines^{4, 40}. Therefore, the observed reduction in serum total IgE and OVA-specific IgE by andrographolide and DDAG in our asthma model may be contributed by its inhibitory effect on B cell activation via inhibition of NF-κB activation, and on IL-4- and IL-13-mediated class switching to IgE.

A family of chitinase proteins including AMCase, Ym1, Ym2 and YKL-40 has recently been found to be markedly elevated in allergic airway inflammation in human and in mouse asthma models²⁶⁻²⁸. They are mainly expressed in airway epithelium and alveolar macrophages. AMCase level is increased in a mouse asthma model and in asthmatic subjects in an IL-13-dependent manner²⁶. When given intratracheally, IL-13 elevates Ym1 and Ym2 levels in BAL fluid from mice in vivo⁵³. Besides, YKL-40 serum level correlates positively with asthma severity, airway remodeling and deterioration of pulmonary function in asthmatic subjects²⁸. Overall, chitinases may play a role in airway inflammation and remodeling. Our data show that andrographolide markedly down-regulated AMCase, Ym2 and YKL-40 mRNA expression in the lungs of OVA-challenged mice. These may be a consequence of the major drop in IL-4 and IL-13 levels in the airways with andrographolide or DDAG treatment and may contribute to the diminished pulmonary eosinophilia.

It is believed that inflammatory mediators released during the allergic inflammation play a critical role in AHR development⁵⁴. We report here that DDAG significantly inhibited OVA-induced AHR to increasing concentrations of methacholine. It has been established that IL-5 plays a critical role in AHR by mobilizing and activating esoinophils, leading to the release of pro-inflammatory products such as major basic protein and cysteinyl-leukotrienes which are closely associated with AHR^{5, 7}. In addition, IL-4 and IL-13 have been shown to induce AHR in mouse asthma models in which cysteinyl-leukotrienes have been implicated in AHR^{6, 55-56}. Moreover, IgE mediated mast cell activation may contribute to AHR by producing a wide array of inflammatory mediators and cytokines^{4,40}. The increase in airway hyperresponsiveness in OVA challange mice may be due to the increased in degranulating mast cell counts found in the lung tissue as the mast cell mediators directly constrict smooth muscle cells and potentiate their constrictor response⁵⁷. Thus, the observed reduction of AHR by DDAG may be associated with the reduction in Th2 cytokine production, tissue eosinophilia, serum IgE level and mast cell degranulation.

Corticosteroids may regulate gene expression in several ways. At clinical dosage, glucocorticoid receptors (GRs), after activation by corticosteroids, translocate to the nucleus and bind to coactivators of NF-κB and recruit histone deacetylase (HDAC)2 to reduce histone acetylation that leads to suppression of these activated inflammatory genes⁵⁹. The main effect of glucocorticoids is to suppress inflammatory transcription factors activities.

Nuclear factor κB activation inhibition by compounds having formula I such as andrographolide or DDAG or the others described herein hold positive prospect in treating patients with glucocorticoid resistance.

[099]. As high dosage oral steroids for long periods is associated with severe side effects, steroid-sparing treatments have been sought after. Even though the anti-inflammatory activities that andrographolide or DDAG exert are less than corticosteroid and nonsteroidal drugs the ability of andrographolide or DDAG to reduce airway inflammation, airway hyperesponsiveness and reduce IgE level led to the speculation that andrographolide or DDAG might be used to control severe, poorly controlled bronchial asthma.

### Combination Therapy

We also examined the therapeutic effect of DDAG and low dose of glucocorticoid in asthma mouse model. We report that DDAG and Steroid can significantly reduced inflammation even at the lowest dose of DDAG and low dose of Steroid combination. By comparing with DDAG alone or Dexamethasone alone, aside from IL-4, IL-5 and IL-13, the DDAG and Dexamethasone combination also significantly decrease the Eotaxin level. Eotaxin recruits eosinophils by inducing their chemotaxis. Thus, therapeutic use of DDAG in combination with a low dose steroid can reduce the dosage of corticosteroid needed for administration, thereby reducing the possible side effect cause by high dosage of glucocorticoid administration.

### Methods

### Animals

Female BALB/c mice (Interfauna, East Yorkshire, UK), were housed in appropriate cages (maximum 4 mice/cage) at the Animal Holding Unit (AHU), NUS. The temperature and humidity of the housing environment were maintained at approximately 22° C. and 55% respectively. Commercial mouse feed and water were provided ad libitum. The beddings were changed three times a week to ensure cleanliness of the living conditions. The handling of mice and the experiments carried out strictly followed the guidelines approved by the Institutional Animal Care and Use Committee (IACUC) of NUS, Singapore.

### Systemic OVA Sensitization

All mice were acclimatized for at least one week before sensitization. A sensitization mixture was prepared by dissolving 20 μg of OVA and 4 mg of Al(OH)₃ in 0.1 ml of Saline. The murine asthma model was developed by sensitizing the mice with 0.1 ml of sensitization mixture on Day 0 and Day 14 using intraperitoneal (i.p.) injections.

### Airway Challenge

The sensitized mice were subjected to airway challenge to produce both the control model as well as the asthma model. A challenge mix was prepared by dissolving 0.15 g OVA into 15 ml of saline solution. The mice were then challenged with aerosolized 1% OVA for 30 minutes on Day 22, 23, 24 to stimulate asthmatic response. A negative control for the asthma model was developed by performing saline challenge on several mice. In this case, 15 ml of saline was used as the challenge mix.

An ultrasonic nebulizer (Ultra-Neb™ 2000 from DeVilbiss Healthcare Inc, USA) was employed to perform the challenge process. The nebulizer aerosolized the challenge mix and pumped the aerosol mist (particle size<5 μm) into an adjacent aerosol chamber where the mice were placed. Mice inhaling aerosolized 1% OVA would be re-exposed to the antigen in the airway, generating an immune response in the airway that mimics asthma pathogenesis and thus creating an ideal model for asthma.

### Preparation of Andrographis Paniculata Extract

1000 grams of Andrographis paniculata aerial part (biomass) are covered with 3.2 liters of 70% ethanol at 65° for 3 hours in a static percolator. Then the percolate is recovered and the biomass is extracted 5 times again under the same conditions, but using 2.6 liters of solvent per extraction, so obtain approximately 15.2 liters of percolate. The combined percolates are filtered and concentrated by a rotary evaporator at 60° under reduced pressure. The extract is dried at 60° under reduced pressure for one night. This extract has a total dry residue of 90.9 g, the yield vs starting material being 10.1 w/w. The Andrographolide HPLC content is 22.38%. Alternatively the andrographolide or DDAG may be purchased commercially from Sigma, St. Louis, Mo.

### Drug Treatment to Mice

Andrographolide or DDAG alone (0.1, 0.5, and 1 mg/kg; Sigma, St. Louis, Mo.) or vehicle (1% dimethyl sulfoxide [DMSO]) in 0.1 ml saline was given by intraperitoneal injections 2 hour before and 10 hours after each OVA aerosol challenge. Andrographolide or 14-deoxy-11,12-didehydroandrographolide (DDA) were prepared from stock. DMSO was used as a solvent to dissolve Andrographolide or DDA. A stock solution of 10 mg/ml was prepared and stored at −20° C. When used, the stock solution was thawed and diluted with Saline solution to form three different drug concentrations: 0.01 mg/ml, 0.05 mg/ml and 0.1 mg/ml. The preparation is shown in Table 1.

TABLE 1
Preparation of different doses of andrographolide or 14-
deoxy-11,12-didehydroandrographolide from stock solution.

| Volume from | | | | |
|---|---|---|---|---|
| 10 mg/ml | Volume of | Total | Final DMSO | Final DDA |
| stock solution | Saline | Volume | concentration | concentration |
| (μl) | (μl) | (μl) | (%) | (mg/ml) |
| 1 | 999 | 1000 | 0.1 | 0.01 |
| 5 | 995 | 1000 | 0.5 | 0.05 |
| 10 | 990 | 1000 | 1.0 | 0.1 |

There were six treatment groups in this study as presented in Table 2. Group A (Saline) consisted of mice sensitized with OVA and challenged with Saline, that served as the negative control group. Group B (OVA) consisted of positive murine asthma model that were OVA-sensitized and OVA-challenged. Group C (DMSO) consisted of mice that were OVA-sensitized, OVA challenged and given intraperitoneal (i.p.) injections of 1% DMSO 2 hours before challenge. This group served as a vehicular control and a negative control for the drug.

Group D (0.1 mg/kg DDA) consisted of mice that were OVA-sensitized, OVA-challenged and given intraperitoneal (i.p.) injections of 0.1 mg/kg andrographolide or DDAG 0.1 mg/kg 2 h before challenge and 10 h later. Group E (0.5 mg/kg DDAG) consisted of mice that were OVA-sensitized, OVA-challenged and given intraperitoneal (i.p.) injections of 0.5 mg/kg andrographolide or 0.5 mg/kg DDAG 2 h before challenge and 10 h later. Group F (1.0 mg/kg andrographolide or 1.0 mg/kg DDAG) consisted of mice that were OVA-sensitized, OVA challenged and given intraperitoneal (i.p.) injections of 1.0 mg/kg DDA 2 h before challenge and 10 h later.

TABLE 2
Different control and treatment groups
(A-F) developed in the study.

| | | Treatment 2 h | | |
|---|---|---|---|---|
| | Sensitization | before Challenge | Challenge | |
| | (Day 0 and | and 10 h later | (Day | Purpose of |
| Group | Day 14) | (Day 22-24) | 22-24) | Treatment |
| A | OVA | — | Saline | (−) model |
| | | | | control |
| B | OVA | — | OVA | (+) model |
| | | | | control |
| C | OVA | 1% DMSO | OVA | Vehicle control |
| D | OVA | 0.1 mg/kg DDA or | OVA | Drug |
| | | andrographolide | | Treatment |
| E | OVA | 0.5 mg/kg DDA or | OVA | Drug |
| | | andrographolide | | Treatment |
| F | OVA | 1.0 mg/kg DDA or | OVA | Drug |
| | | andrographolide | | Treatment |

### Bronchoalveolar Lavage (BAL) Fluid

Bronchoalveolar lavage (BAL) was conducted on Day 25, 24 hours after the final OVA/saline challenge. Mice were anaesthetized by 0.3 ml i.p. injections of an anesthetic cocktail (ketamine: medetomidine: H2O=3:4:33, Parnell, Alexandria NSW, Australia & Pfizer, Auckland, New Zealand) with 27 G½ sterile needle (PrecisionGlide®). Mice were sacrificed by cervical dislocation 5 minutes after anesthetization. 25 G½ sterile needles (PrecisionGlide®) were used for blood extraction. When withdrawing the blood, the needles were pierced diagonally into the wall of the left ventricles, and the aspiration should be slow so that the blood flow did not collapse. Tracheotomy was then performed and a small transverse incision was made on the exposed trachea. Blunt needles (20 G), which were connected to 1 ml sterile syringes, were inserted into the trachea through this incision and 0.5 ml of ice-cold PBS (4° C.) was instilled thrice into the lungs (0.5 ml×3). About 1.2-1.4 ml of BAL fluid was retrieved from each mouse and was kept in −80° C. for subsequent experiments.

### Serum Collection

Blood collected from the hearts were allowed to clot for at least 4 h. Centrifugation at 3000 rpm for 5 min at 4° C. was then performed on all blood samples. The supernatant, which is the serum, was extracted carefully and stored at −80° C. The samples were kept for ELISA.

### Total Cell Count

BAL fluid collected from the lungs of the mouse was centrifuged at 3000 rpm for 5 min at 4° C. The supernatant was collected and stored at −80° C. The pellet was resuspended in 200 μl of 0.875% NH₄Cl (8.75 mg NH₄Cl in 1 ml MilliQ water) and incubated for 5 minutes at room temperature to remove unwanted erythrocytes. The cell suspension was then centrifuged at 3000 rpm for 5 min at 4° C. The supernatant was discarded and the pellet containing inflammatory cells was resuspended in 200 μl of RPMI with 1% BSA (10 mg BSA in 1 ml RPMI). The total number of viable cells was enumerated using a haemocytometer (10 μl of 0.4% trypan blue: 10 μl of cell suspension), under a microscope at 200×magnification.

### Differential Cell Count

Following the total cell count, dilutions were performed on the BAL fluid collected with the RPMI/BSA solution (1×10⁵ cells per 150 μl of RPMI/BSA). Cytocentrifugation was then carried out on all samples using a Cytospin centrifuge (Thermo Shandon, Pittsburgh, USA) at 600 g for 10 minutes to fix the cells on glass slides. Smears of the infiltrating inflammatory cells were allowed to air-dry and then stained using Liu staining (modified Wright staining). In Liu staining, cytospin slides were stained with 800 μl of Liu A for 30 seconds and then followed by 1600 μl of Liu B for 90 seconds. Slides were left to dry overnight and glass cover slips were mounted onto the stain. Differential cell count was then performed on a minimum of 500 cells under the microscope (1000×magnification) for each cytospin slide. Four types of inflammatory cells namely macrophages, eosinophils, neutrophils and lymphocytes were identified and their respective ratio was enumerated based on the staining outcome and distinctive morphological features. The absolute number of each inflammatory cell was calculated.

### Test Treatments

DDAG alone (0.1, 0.5, and 1 mg/kg; Sigma, St. Louis, Mo.) or vehicle (2% dimethyl sulfoxide [DMSO]) in 0.1 ml saline was given by intraperitoneal injections 2 hour before and 10 hours after each OVA aerosol challenge. In the combinational experiments, DDAG (0.1 mg/kg) in combination with Dexamethasone (0.05 mg/kg) was given intraperitoneally. In both experimental setting, saline aerosol was used as a negative control. Animal experiments were performed according to the Institutional guidelines for Animal Care and Use Committee of the National University of Singapore.

### Cytokine and Chemokine Levels in BAL Fluid

Levels of IL-4, 11-5, IL-13, eotaxin and IFN-γ in the BAL fluid were measured (IL-4, IL-5 and IFN-γ from BD Biosciences Pharmingen, San Diego, Calif., USA; IL-13 and eotaxin from R&D Systems, Minneapolis, Minn., USA). The kits were obtained from two different manufacturers and thus the protocols differed in certain steps. In brief, 50 μl of coating capture antibody in respective coating buffers (pH 9.5, 0.1 M sodium carbonate for IL-4, IL-5 and IFN-γ; 1×PBS for IL-13 and eotaxin) was coated to 96-well ELISA plate (NUNC, Denmark). The plate was sealed with parafilm and incubated overnight at respective temperature (4° C. for IL-4, IL-5 and IFN-γ and room temperature for IL-13 and eotaxin). Next day, each well was washed with washing buffer (PBS with 0.05% Tween-20) and blocked with 200 μl blocking buffer (PBS with 10% FBS for IL-4, IL-5 and IFN-γ; 1×PBS with 1% BSA and 5% sucrose for IL-13 and eotaxin) for 2 hours at room temperature. After blocking, 50 μl of standards and BAL fluid samples were loaded into respective wells, and incubated for 2 h at room temperature. Several washings were done to remove unbound molecules. The plate was incubated with biotinylated-detection antibody with HRP for 1 h (BD OptEIA™ Kit) or with biotinylated detection antibody for 1 h followed by HRP for 45 min (R&D Kit). After washing, 50 μl of TMB peroxidase substrate (solution A: solution B=9:1) was added into each well, and incubated for 30 min in dark. Lastly, 50 μl of stopping solution (1 M H₂SO₄) was added to stop the reaction. The optical density of each well in the plate was read at 450 nm with λ correction at 570 nm. Detection limits of the respective kits are as follow: 4 pg/ml for IL-4 and IL-5; 15.6 pg/ml for IL-13 and IFN-γ; and 2 pg/ml for eotaxin.

### Bronchoalveolar Lavage Fluid and Serum Analysis

Mice were anesthetized 24 hours after the last aerosol challenge and bronchoalveolar lavage (BAL) was performed as described above. BAL fluid total and differential cell counts, and cytokine and chemokine levels were determined as described above. Blood was collected by cardiac puncture, and serum levels of total IgE and OVA-specific IgE, IgG1, and IgG2a levels were determined.

Levels of total IgE and OVA-specific IgE, IgG 1 , and IgG2a in serum were measured. 96-well ELISA plate coated with either 50 μl of capture antibody (for total IgE, 1:250 dilution with 1 M Na₂CO₃) or 50 μl of 20 μg/ml OVA (for OVA-specific IgE, IgG1, and IgG2a) overnight at 4° C. Next day, the plate was washed with washing buffer (PBS with 0.05% Tween-20 for total IgE; PBS with 0.1% Tween-20 for OVA-specific IgE) and blocked with 300 μl 10% FBS in PBS for 2 hr at room temperature. After blocking, standards (only for total IgE) and serum samples were loaded into respective wells and incubated for 2 h. Followed by washing, respective detective antibodies were added and incubated for 1 h, followed by 45 min incubation of HRP-conjugate antibody for 45 min in the dark. Substrates were then added for 30 min in the dark with mild shaking. Lastly, 50 μl of stopping solution (1 M H₂SO₄) was added to stop the reaction. The optical density of each well in the plate was read at 450 nm with λ correction at 570 nm. The detection limit for total IgE was 2 ng/ml.

### Histologic Analysis

Lungs were fixed in 10% neutral formalin, paraffinized, cut into 6-μm sections, and stained with hematoxylin and eosin (H&E) for examining cell infiltration and with periodic acid-Schiff stain (PAS) for measuring mucus production. Quantitative analysis was performed blinded as described below. Mast cells were detected in lung tissue using toluidine-blue staining, and the number of mast cells was counted in paraffin sections. The percentage of degranulated mast cells in the lung was calculated by counting the number of cells with 10% of extrusion of granules.

### Qualitative Analysis and Scoring Criteria

For both H&E and PAS staining, bronchioles selected for analysis were of similar structure and sizes, with clear morphology presentations and minimum disruptions of the surrounding tissues that could be formed during sectioning. The scoring of inflammatory and globlet cells was performed in 2-4 preparations of each mouse and mean scores were calculated from 4-5 mice. The scoring criteria is summarized in Table 3. Peribronchial cell counts were performed in a blind manner and scored as known in the art. A five-point scoring system (0-4) was adapted: 0, no cells; 1, a few cells; 2, a ring of cells one cell layer deep; 3, a ring of cells two to four cells deep; 4, a ring of cells more than four cells deep. Goblet cell hyperplasia in the airway epithelium was assessed blind and scored according to the percentage of PAS-positive mucus-producing cells. A five-point (0-4) grading system was as follows: 0, no goblet cells; 1, <25%; 2, 25-50%; 3, 50-75%; 4, >75%. Briefly, to determine the severity of inflammatory cell infiltration, peribronchial cell counts were performed blind based on a 5-point scoring system: 0, no cells; 1, a few cells; 2, a ring of cells 1 cell layer deep; 3, a ring of cells 2-4 cells deep; 4, a ring of cells of >4 cells deep. To determine the extent of mucus production, goblet cell hyperplasia in the airway epithelium was quantified blind using a 5-point grading system: 0, no goblet cells; 1, <25%; 2, 25-50%; 3, 50-75%; 4, >75%. Scoring of inflammatory cells and goblet cells was performed in at least 3 different fields for each lung section. Mean scores were obtained from 4 animals. *Significant difference from DMSO control, P<0.05.

TABLE 3
Scoring systems for both H&E staining and
PAS staining of the sectioned lung samples.

| Score | H&E Staining | PAS Staining |
|---|---|---|
| 0 | no cells | 0% |
| 1 | a few cells | <25% |
| 2 | a ring of cells 1 layer deep | 25-50% |
| 3 | a ring of cells 2-4 layers deep | 50-75% |
| 4 | a ring of cells >4 layers deep | >75% |

### Measurements of Airway Hyper-Responsiveness (AHR)

Mice were anesthetized and tracheotomy was performed as described above. The internal jugular vein was cannulated and connected to a microsyringe for intravenous methacholine administration. Airway resistance (RI) and dynamic compliance (Cdyn) in response to increasing concentrations of mechacholine were recorded using a whole body plethysmograph chamber (Buxco, Sharon, Conn.) and ventilated mechanically by a ventilator via the tube that was inserted into the trachea at a tidal volume of 200 μl/breadth and a breathing rate of 150/min. Airflow and pressure changes were detected by respective transducers, recorded and analyzed by Biosystem XA software (Buxco, Sharon, Conn., USA). Results are expressed as a percentage of the respective basal values in response to phosphate buffered saline (PBS).

### Cell Cultures

To determine the effects of andrograhpolide on OVA-specific immune responses in lymphocytes, thoracic lymph node cells were grown in bronchial epithelial bulletkit medium (Cambrex BioScience, Walkersvile, Md., USA), supplemented with bovine pituitary extract (2 ml), hydrocortisone (0.5 ml), recombinant human EGF (0.5 ml), epinephrine (0.5 ml), transferrin (0.5 ml), insulin (0.5 ml), retinoic acid (0.5 ml), triiodothyronine (0.5 ml), gentamycin sulfate (50 μg/ml), and amphotericin B (50 ng/ml). The cells were incubated in 5% CO₂ incubator at 37° C., and subcultured at 80% to 85% confluency. Cells were exposed to 200 μg/ml OVA for 72 hours. Concanavalin A (Con A, 10 μug/ml) was used as a positive control. Supernatants from parallel triplicate cultures were analyzed for cytokine levels by ELISA. Normal human bronchial epithelial cells were cultured in optimized bronchial epithelial bulletkit medium with supplements (Lonza, Basel, Switzerland). Cells were pretreated with 30 μM andrographolide or vehicle (0.01% DMSO) 4 hours before stimulation with 10 ng/ml tumor necrosis factor (TNF)-α. Total and nuclear proteins, and mRNA were extracted from cells at specified time intervals.

### Immunoblotting,

Lung and cell culture nuclear proteins (10 mg per lane) were isolated. Lysates were incubated on ice for 30 min before centrifugation (18,000 g for 5 min). The supernatants were collected, and protein concentrations were determined using a BCA protein assay kit. 28 μl of the protein measured were mixed with 7 μl 5× sample buffer and boiled at 95° C. for 5 min. 10% SDS-PAGE gel was set as known in the art. The gel was placed in a Trans-Blot tank (Bio-Rad Laboratories, Hercules, Calif.). 30 μl of the sample mixtures and Pre-stained SDS Page Marker (Bio-Rad Laboratories, Hercules, Calif.) were loaded into respective wells and ran at 100V for 2 hours. The proteins were then transferred to a PVDF membrane using a semi-dry transblotter (ATTO Corp, Tokyo, Japan). The PVDF membrane was blocked with 5% non-fat milk in Tween-20 Tris buffered saline (TTBS) for 2 hours and probed with various primary antibodies in 1% non-fat milk in TTBS (1:2,000 for anti-rabbit antibodies; 1:5,000 for anti-mouse antibodies; 1:10,000 for β-actin) overnight at 4° C. The PVDF was then washed with TTBS for 10 times (2-3 min each), and incubated in HRP- or AP-conjugated anti-mouse or anti-rabbit antibodies for 1 hour. The membrane was again washed with TTBS. For HRP-conjugated antibody, 1 ml each of HRP substrate 1 and substrate 2 were added onto the membrane. For AP-conjugated antibody, 7.5 ml of AP substrates (300 μl AP 25×Buffer, 75 μl Reagent A, 75 μl Reagent B, 7.5 ml water) was added per gel. The data was then developed in the dark on hyperfilms using an ECL reagent. Immunoblots were probed with anti-p65, anti-phospho-p65 (Ser536), and anti-TATA binding protein (TBP, Abcam, Cambridge, UK).

mRNA Expression

Lungs were isolated from the thoracic cavity 24 hours after the last OVA or saline challenge, and stored in RNAlater. The samples were first incubated at −40° C. overnight for the RNAlater to permeate into the lung tissues to stabilize the RNA. The samples were then stored at −80° C. Before RNA isolation, lung tissues were thawed at −4° C. They were then removed from the RNAlater and immersed in 1 ml Trizol solution. Then, homogenization was performed using a homogenizer (SilentCrusher M, Heidolph Elektro GmbH & Co., Kelheim, Genman). All samples were placed on ice to prevent RNA degradation. The homogenates were then centrifuged at 12,000 g for 10 min at 4° C. The clear RNA containing supernatant was then decanted, incubated for 5 min in room temperature, and 0.2 ml of chloroform was added. All tubes were shaken vigorously for 15 seconds and incubated at room temperature for 3 minutes before being centrifuged at 12,000 g for 15 min at 4° C. About 500 μl of the colourless upper aqueous layer containing RNA, was decanted. 0.5 ml of isopropanol was added and mixed for 30 sec, before being incubated at room temperature for 10 minutes. The tubes were then centrifuged at 12000 rpm for 15 min at 4° C. The supernatant was discarded and 1 ml of 75% ethanol was added to the RNA pellet. Centrifugation at 8500 rpm for 5 minutes at 4° C. was then performed to wash the pellet. The supernatant was again discarded and the washed RNA pellet was air-dried at room temperature for 10 min. The dried RNA pellet was dissolved in 100 μl of ribonuclease-free DEPC water and incubated at 55° C. for 10 minutes. The amount and purity of RNA present in the sample was quantified using the spectrophotometer (NanoDrop ND-1000 from Thermo Risher

Scientific Inc., Waltham, Mass., USA). Both the A₂₆₀/A₂₈₀ (DNA/protein) and A₂₆₀/A₂₃₀ (DNA/organic contaminants) ratios were recorded as an indication of the purity of the RNA extracted. An acceptable level of purity for both A₂₆₀/A₂₈₀ and A₂₆₀/A₂₃₀ readings should be about 1.8 to 2.0.

Reverse transcription was performed to synthesize single-stranded complementary DNA (cDNA) templates from the RNA extracted. Volume containing 1 μg total RNA was calculated based on the nucleic acid concentration measured from the spectrophotometer, and was topped up to 20 μl with DEPC water. 10.58 μl of master mix was then added to each sample. Then, cDNA was synthesized from 1 μg of RNA using a multiwall thermal cycler (GeneAmp PCR system 2700 from Applied Biosystems, Foster City, Calif., USA) by bringing the reaction volume to 95° C. for 10 minutes and 42° C. for 30 minutes.

PCR amplifications were then performed using the multiwell thermal cycler on 1 μl cDNA template in a 25 μl reaction volume. The reaction volume contained 10.5 μl nuclease-free water, 0.5 μl forward primer (10 μM), 0.5 μl reverse primer (10 μM) and 12.5 μl 2×PCR master mix (50 units/ml TaqDNA polymerase, 400 μM dATP, 400 μM dGTP, 400 μM dCTP, 400 μM dTTP and 3 mM MgCl₂). Primers for inflammatory biomarkers are shown in Table 4.

TABLE 4
PRIMER SETS FOR REVERSE TRANSCRIPTASE-POLYMERASE CHAIN REACTION ANALYSIS
Sequences

| Targets | Forward | Reverse |
|---|---|---|
| AMCase | 5′-TGGGTTCTGGGCCTACTATG-3′ | 5′-GCTTGACAATGCTGCTGGTA-3′ |
| (mouse) | | |
| Ym1 | 5′-CTGGAATTGGTGCCCCTACA-3′ | 5′-CAAGCATGGTGG TTTTACAGGA-3 |
| (mouse) | | |
| Ym2 | 5′-CAGAACCGTCAGACATTCATTA-3′ | 5′-ATGGTCCTTC CAGTAGG TAATA-3′ |
| (mouse) | | |
| YKL-40 | 5′-GTACAAGCTGGTCTGCTACT-3′ | 5′-GTTGGAGGCAATCTCGGAAA-3′ |
| (mouse) | | |
| E-selectin | 5′-AACGCCAGAACAACAATTCC-3′ | 5′-TGAATTGCCACCAGATGTGT-3′ |
| (mouse) | | |
| MCP-1 | 5′-GATCTCAGTGCAGAGGCTCG-3′ | 5′-TGCTTGTCCAGGTGGTCCAT-3′ |
| (human) | | |
| COX-2 | 5′-GGAGAGACTATCAAGATAGT-3′ | 5′-ATGGTCAGTAGACTTTTACA-3′ |
| Muc5ac | 5′-GAGTGACATTGCAGGAAGCA-3′ | 5′-CAGAGGACAGGAAGGTGAGC-3′ |
| (mouse) | | |
| iNOS | 5′-GTCAACTGCAAGAGAACGGAGAC-3′ | 5′-GAGCTCCTCCAGACGGGTAGGCTTG-3′ |
| (mouse) | | |
| IL-8 | 5′-ATGACTTCCAAGCTGGCCGTGGCT-3′ | 5′-TCTCAGCCCTCTTCAAAAACTTCTC-3′ |
| (human) | | |
| IL-17 | 5′-CCGCAATGAAGACCCTGATAGA-3′ | 5′-CAGCATCTTCTCGACCCTGAAA-3′ |
| IL-33 | 5′-GATGGGAAGAAGGTGATGGGTG-3′ | 5′-TTGTGAAGGACGAAGAAGGC-3′ |
| RANTES | 5′-ATGAAGGTCTCCGCGGCACGCCT-3′ | 5′-CTAGCTCATCTCCAAAGAGTTG-3′ |
| (human) | | |
| VCAM-1 | 5′-CAAGGGTGACCAGCTCATGAA-3′ | 5′-TGTGCAGCCACCTGAGATCC-3′ |
| (mouse) | | |
| GADPH | 5′-GGCAAATTCAACGGCACA-3′ | 5′-GTTAGTGGGGTCGTCCTG-3′ |
| (mouse) | | |
| β-Actin | 5′-TCATGAAGTGTGACGTTGACATCCGT-3′ | 5′-CCTAGAAGCATTTGCGGTGCACGATG-3′ |
| (both) | | |

### NF-κB DNA-Binding

Nuclear proteins were also analyzed for NF-κB DNA-binding activity using the TransAM p65 transcription factor assay kit (Active Motif, Carlsbad, Calif.).

### Statistical Analysis

Data are presented as means±SEM. One-way ANOVA followed by Dunnett's test was used to determine significant differences between treatment groups. Significant levels were set at P<0.05.

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (eg size, concentration etc). A range of values will be understood to include all values within the range, including the values defining the range, and values adjacent to the range which lead to the same or substantially the same outcome as the values immediately adjacent to that value which defines the boundary to the range.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

While the invention has been described with reference to specific methods and embodiments, it will be appreciated that various modifications and changes may be made without departing from the invention.

### REFERENCE

1. Galli S J, Tsai M, Piliponsky A M. The development of allergic inflammation. Nature. Jul. 24 2008;454(7203):445-454.
2. Medoff B D, Thomas S Y, Luster A D. T cell trafficking in allergic asthma: the ins and outs. Annu Rev Immunol. 2008;26:205-232.
3. Li-Weber M, Krammer P H. Regulation of IL4 gene expression by T cells and therapeutic perspectives. Nat Rev Immunol. July 2003;3(7):534-543.
4. Galli S J, Grimbaldeston M, Tsai M. Immunomodulatory mast cells: negative, as well as positive, regulators of immunity. Nat Rev Immunol. June 2008;8(6):478-486.
5. Takatsu K, Nakajima H. IL-5 and eosinophilia. Curr Opin Immunol. June 2008;20(3):288-294.
6. Wills-Karp M. Interleukin-13 in asthma pathogenesis. Immunol Rev. December 2004;202:175-190.
7. Hogan S P, Rosenberg H F, Moqbel R, et al. Eosinophils: biological properties and role in health and disease. Clin Exp Allergy. May 2008;38(5):709-750.
8. Berend N, Salome C M, King G G. Mechanisms of airway hyperresponsiveness in asthma. Respirology. September 2008;13(5):624-631.
9. Zhou J, Lu G D, Ong C S, Ong C N, Shen H M. Andrographolide sensitizes cancer cells to TRAIL-induced apoptosis via p53-mediated death receptor 4 up-regulation. Mol Cancer Ther. July 2008;7(7):2170-2180.
10. Liang F P, Lin C H, Kuo C D, Chao H P, Fu S L. Suppression of v-Src transformation by andrographolide via degradation of the v-Src protein and attenuation of the Erk signaling pathway. J Biol Chem. Feb. 22 2008;283(8):5023-5033.
11. Negi A S, Kumar J K, Luqman S, Shanker K, Gupta M M, Khanuja S P. Recent advances in plant hepatoprotectives: a chemical and biological profile of some important leads. Med Res Rev. September 2008;28(5):746-772.
12. Iruretagoyena M I, Tobar J A, Gonzalez P A, et al. Andrographolide interferes with T cell activation and reduces experimental autoimmune encephalomyelitis in the mouse. J Pharmacol Exp Ther. January 2005;312(1):366-372.
13. Wang Y J, Wang J T, Fan Q X, Geng J G. Andrographolide inhibits NF-kappaBeta activation and attenuates neointimal hyperplasia in arterial restenosis. Cell Res. November 2007;17(11):933-941.
14. Xia Y F, Ye B Q, Li Y D, et al. Andrographolide attenuates inflammation by inhibition of NF-kappa B activation through covalent modification of reduced cysteine 62 of p50. J Immunol. Sep. 15 2004;173(6):4207-4217.
15. Schacke, H., Docke, W. D., and Asadullah, K. (2002). Mechanisms involved in the side effects of glucocorticoids. Pharmacology and Therapeutics. 96: 23 -43.
16. Chiou WF C C, Lin J J. Mechanisms of suppression of inducible nitric oxide synthase (iNOS) expression in RAW 264.7 cells by andrographolide. Br J PharmacoL 2000; 129:1553-1560.
17. Chiou W F L J, Chen C F. Andrographolide suppresses the expression of inducible nitric oxide synthase in macrophage and restores the vasoconstriction in rat aorta treated with lipopolysaccharide. Br J Pharmacol. 1998;125:327-334.
18. Alan Balmain J D C. Minor diterpenoid constituents of Andrographis paniculata Nees. Perkin Translations 1. 1973:1247 -1251.
19. Suebsasana S, Pongnaratom P, Sattayasai J, Arkaravichien T, Tiamkao S, Aromdee C. Analgesic, antipyretic, anti-inflammatory and toxic effects of andrographolide derivatives in experimental animals. Arch Pharm Res. September 2009;32(9):1191-1200.
20. Deng W L, Nie, R. J., and Liu, J. Y. Comparison of pharmacological effect of four andrographolides. Yaoxue Tongbao. 1982;17:195-198.

21. Barnes P J, Adcock I M. How do corticosteroids work in asthma? Ann Intern Med. Sep. 2 2003;139(5 Pt 1):359-370.
22. Ito K, Chung K F, Adcock I M. Update on glucocorticoid action and resistance. J Allergy Clin Immunol. March 2006;117(3):522-543.
23. Duan W, Chan J H, Wong C H, Leung B P, Wong WS. Anti-inflammatory effects of mitogen-activated protein kinase kinase inhibitor U0126 in an asthma mouse model. J Immunol. Jun. 1 2004;172(11):7053-7059.
24. Pushparaj P N, Tay H'Ng S C, et al. The cytokine interleukin-33 mediates anaphylactic shock. Proc Natl Acad Sci USA. Jun. 16 2009;106(24):9773-9778.
25. Kelly M, Hwang J M, Kubes P. Modulating leukocyte recruitment in inflammation. J Allergy Clin Immunol. July 2007;120(1):3-10.
26. Zhu Z, Zheng T, Homer R J, et al. Acidic mammalian chitinase in asthmatic Th2 inflammation and IL-13 pathway activation. Science. Jun. 11 2004; 304(5677):1678-1682.
27. Zhao J, Zhu H, Wong C H, Leung K Y, Wong W S. Increased lungkine and chitinase levels in allergic airway inflammation: a proteomics approach. Proteomics. July 2005;5(11):2799-2807.
28. Chupp G L, Lee C G, Jarjour N, et al. A chitinase-like protein in the lung and circulation of patients with severe asthma. N Engl J Med. Nov. 15 2007; 357(20):2016-2027.
29. Morcillo E J, Cortijo J. Mucus and MUC in asthma. Curr Opin Pulm Med. January 2006;12(1):1-6.
30. Smith D E. IL-33: a tissue derived cytokine pathway involved in allergic inflammation and asthma. Clin Exp Allergy. Nov. 3 2009.
31. Nembrini C, Marsland B J, Kopf M. IL-17-producing T cells in lung immunity and inflammation. J Allergy Clin Immunol. May 2009;123(5):986-994; quiz 995-986.
32. Brightling C, Berry M, Amrani Y. Targeting TNF-alpha: a novel therapeutic approach for asthma. J Allergy Clin Immunol. January 2008;121(1):5-10; quiz 11-12.
33. Vroling A B, Duinsbergen D, Fokkens W J, van Drunen C M. Allergen induced gene expression of airway epithelial cells shows a possible role for TNF-alpha. Allergy. November 2007;62(11):1310-1319.
34. Newton R, Holden N S, Catley M C, et al. Repression of inflammatory gene expression in human pulmonary epithelial cells by small-molecule IkappaB kinase inhibitors. J Pharmacol Exp Ther. May 2007;321(2):734-742.
35. Gagliardo R, Chanez P, Mathieu M, et al. Persistent activation of nuclear factorkappaB signaling pathway in severe uncontrolled asthma. Am J Respir Crit Care Med. Nov. 15 2003;168(10):1190-1198.
36. Hart L A, Krishnan V L, Adcock I M, Barnes P J, Chung K F. Activation and localization of transcription factor, nuclear factor-kappaB, in asthma. Am J Respir Crit Care Med. November 1998;158(5 Pt 1):1585-1592.
37. Pantano C, Ather J L, Alcorn J F, et al. Nuclear factor-kappaB activation in airway epithelium induces inflammation and hyperresponsiveness. Am J Respir Crit Care Med. May 1 2008;177(9):959-969.
38. Poynter M E, Cloots R, van Woerkom T, et al. NF-kappa B activation in airways modulates allergic inflammation but not hyperresponsiveness. J Immunol. Dec. 1 2004;173(11):7003-7009.
39. Schulze-Luehrmann J, Ghosh S. Antigen-receptor signaling to nuclear factor kappa B. Immunity. November 2006;25(5):701-715.
40. Klemm S, Ruland J. Inflammatory signal transduction from the Fc epsilon RI to NFkappa B. Immunobiology. 2006;211(10):815-820.
41. Desmet C, Gosset P, Pajak B, et al. Selective blockade of NF-kappa B activity in airway immune cells inhibits the effector phase of experimental asthma. J Immunol. Nov 1 2004;173(9):5766-5775.
42. Choi I W, Kim D K, Ko H M, Lee H K. Administration of antisense phosphorothioate oligonucleotide to the p65 subunit of NF-kappaB inhibits established asthmatic reaction in mice. Int Immunopharmacol. Dec. 20 2004;4(14):1817-1828.
43. Birrell M A, Hardaker E, Wong S, et al. Ikappa-B kinase-2 inhibitor blocks inflammation in human airway smooth muscle and a rat model of asthma. Am J Respir Crit Care Med. Oct. 15 2005;172(8):962-971.
44. Das J, Chen C H, Yang L, Cohn L, Ray P, Ray A. A critical role for NF-kappa B in GATA3 expression and TH2 differentiation in allergic airway inflammation. Nat Immunol. January 2001;2(1):45-50.
45. Broide D H, Lawrence T, Doherty T, et al. Allergen-induced peribronchial fibrosis and mucus production mediated by IkappaB kinase beta-dependent genes in airway epithelium. Proc Natl Acad Sci USA. Dec. 6 2005;102(49):17723-17728.
46. Schmitz J, Owyang A, Oldham E, et al. IL-33, an interleukin-1-like cytokine that signals via the IL-1 receptor-related protein ST2 and induces T helper type 2-associated cytokines. Immunity. November 2005;23(5):479-490.
47. Li L, Xia Y, Nguyen A, et al. Effects of Th2 cytokines on chemokine expression in the lung: IL-13 potently induces eotaxin expression by airway epithelial cells. J ImmunoL Mar. 1 1999;162(5):2477-2487.
48. Rahman M S, Yamasaki A, Yang J, Shan L, Halayko A J, Gounni A S. IL-17A induces eotaxin-1/CC chemokine ligand 11 expression in human airway smooth muscle cells: role of MAPK (Erk½, JNK, and p38) pathways. J Immunol. Sep. 15 2006;177(6):4064-4071.
49. Kumar A, Takada Y, Boriek A M, Aggarwal B B. Nuclear factor-kappaB: its role in health and disease. J Mol Med. July 2004;82(7):434-448.
50. Justice J P, Crosby J, Borchers M T, Tomkinson A, Lee J J, Lee N A. CD4(+) T celldependent airway mucus production occurs in response to IL-5 expression in lung. Am J Physiol Lung Cell Mol Physiol. May 2002;282(5):L1066-1074.
51. Lora J M, Zhang D M, Liao S M, et al. Tumor necrosis factor-alpha triggers mucus production in airway epithelium through an IkappaB kinase beta-dependent mechanism. J Biol Chem. Oct. 28 2005;280(43):36510-36517.
52. Siebenlist U, Brown K, Claudio E. Control of lymphocyte development by nuclear factor-kappaB. Nat Rev Immunol. June 2005;5(6):435-445.
53. Webb D C, McKenzie A N, Foster P S. Expression of the Ym2 lectin-binding protein is dependent on interleukin (IL)-4 and IL-13 signal transduction: identification of a novel allergy-associated protein. J Biol Chem. Nov. 9 2001;276(45):41969-41976.
54. Cockcroft D W, Davis B E. Mechanisms of airway hyperresponsiveness. J Allergy Clin Immunol. September 2006;118(3):551-559; quiz 560-551.
55. Vargaftig B B, Singer M. Leukotrienes mediate murine bronchopulmonary hyperreactivity, inflammation, and part of mucosal metaplasia and tissue injury induced by recombinant murine interleukin-13. Am J Respir Cell Mol Biol. April 2003;28(4):410-419.
56. Leigh R, Ellis R, Wattie J N, et al. Type 2 cytokines in the pathogenesis of sustained airway dysfunction and airway remodeling in mice. Am J Respir Crit Care Med. Apr. 1 2004;169(7):860-867.
57. Robinson D S. The role of the mast cell in asthma: induction of airway hyperresponsiveness by interaction with smooth muscle? J Allergy Clin Immunol. July 2004;114(1):58-65.

58. Barnes P J. Corticosteroid effects on cell signalling. Eur Respir J. February 2006;27(2):413-426.
59. Ito K, Yamamura S, Essilfie-Quaye S, et al. Histone deacetylase 2-mediated deacetylation of the glucocorticoid receptor enables NF-kappaB suppression. J Exp Med. Jan. 23 2006;203(1):7-13.

1. A method of controlling inflammation in a lung cell comprising administering a dose of formula I. 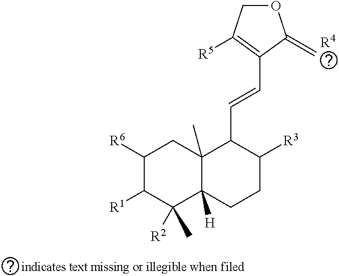
wherein,
R¹ and R² may be selected from a hydroxyl group, a methoxy group, a methylene group, or an ether or ester linked sugar group; hydrogen, substituted or unsubstituted, linear or branched (C₁-C₈) alkyl group such as methyl, ethyl, n-propyl, iso-propyl and the like; aryl group such as phenyl, naphthyl and the like, the aryl group may be substituted; heteroaryl group such as pyridyl, furyl, thiophenyl and the like, the heteroaryl group may be substituted; aralkyl such as benzyl, phenethyl and the like, the aralkyl group may be substituted; heteroaralkyl group such as pyridylmethyl, pyridylethyl, furanmethyl, faranethyl and the like, the heteroaralkyl group may be substituted; (C₂-C₈) alkanoyl group such as ethanoyl, propanoyl, butanoyl and the like, the (C₂-C₈) alkanoyl group may be substituted; (C₃-C₈) alkenoyl group such as propenoyl, butenoyl, pentenyl and the like, (C₃-C₈) alkenoyl group may be substituted; aroyl group such as benzoyl and the like, the aroyl group may be substituted; heteroaroyl group such as pyridyl carbonyl, furyl carbonyl and the like; the heteroaroyl group may be substituted; aralkenoyl group such as phenylpropenoyl, phenylbutenoyl, phenylpentenoyl and the like, the aralkenoyl group may be substituted; aralkanoyl group such as phenylpropanoyl, phenylbutanoyl, phenylpentanoyl and the like, the aralkanoyl group may be substituted; sulfonyl group such as methanesulfonyl, benzenesulfonyl, p-toluenesulfonyl and the like, the sulfonyl group may be substituted.
R³ is selected from a methyl group or a methylene group;
R⁴ is selected from a hydroxyl group or a carbonyl group;
R⁵ is selected from one of the following: a hydroxyl group, an alkyl group, a methoxy group, a methylene group, or an ether or ester linked sugar group;

2. The method of claim 1 wherein the cell is in vitro.

3. The method of claim 1 wherein the cell is in vivo and the formula I is administered to a patient in need of controlling an airway disorder.

4. The method of claim 1 wherein formula I is Andrographolide.

5. The method of claim 1 wherein formula I is 14-deoxy-1 1,12-didehydroandrographolide.

6. The method of claim 1 wherein controlling inflammation comprises controlling asthma.

7. The method of claim 1 wherein controlling inflammation comprises controlling allergenic effects.

8. The method of claim 1 wherein controlling inflammation comprises controlling chronic obstructive pulmonary disease (COPD).

9. A method of treating an airway disorder comprising administering a dose of formula I: 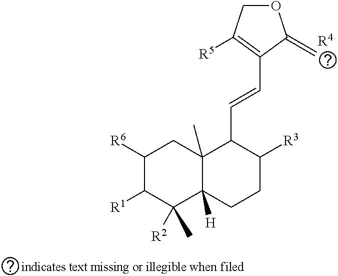
wherein,
R¹ and R² may be selected from a hydroxyl group, a methoxy group, a methylene group, or an ether or ester linked sugar group; hydrogen, substituted or unsubstituted, linear or branched (C₁-C₈) alkyl group such as methyl, ethyl, n-propyl, iso-propyl and the like; aryl group such as phenyl, naphthyl and the like, the aryl group may be substituted; heteroaryl group such as pyridyl, furyl, thiophenyl and the like, the heteroaryl group may be substituted; aralkyl such as benzyl, phenethyl and the like, the aralkyl group may be substituted; heteroaralkyl group such as pyridylmethyl, pyridylethyl, furanmethyl, faranethyl and the like, the heteroaralkyl group may be substituted; (C₂-C₈) alkanoyl group such as ethanoyl, propanoyl, butanoyl and the like, the (C₂-C₈) alkanoyl group may be substituted; (C₃-C₈) alkenoyl group such as propenoyl, butenoyl, pentenyl and the like, (C₃-C₈) alkenoyl group may be substituted; aroyl group such as benzoyl and the like, the aroyl group may be substituted; heteroaroyl group such as pyridyl carbonyl, furyl carbonyl and the like; the heteroaroyl group may be substituted; aralkenoyl group such as phenylpropenoyl, phenylbutenoyl, phenylpentenoyl and the like, the aralkenoyl group may be substituted; aralkanoyl group such as phenylpropanoyl, phenylbutanoyl, phenylpentanoyl and the like, the aralkanoyl group may be substituted; sulfonyl group such as methanesulfonyl, benzenesulfonyl, p-toluenesulfonyl and the like, the sulfonyl group may be substituted.
R³ is selected from a methyl group or a methylene group;
R⁴ is selected from a hydroxyl group or a carbonyl group;
R⁵ is selected from one of the following: a hydroxyl group, an alkyl group, a methoxy group, a methylene group, or an ether or ester linked sugar group;

10. The method of claim 9 wherein formula I is Andrographolide.

11. The method of claim 9 wherein formula I is 14-deoxy-1 1,12-didehydroandrographolide.

12. The method of claim 9 wherein the airway disorder is an asthma exacerbation.

13. The method of claim 9 wherein the airway disorder is COPD

14. The method of claim 1 or 9 further comprising administering a corticosteroid.

15. The method of claim 14 wherein the corticosteroid comprises Dexamethasone, Budesonide, Fluticasone, Ciclesonide, or Beclomethasone Dipropionate.

16. A compound of formula I 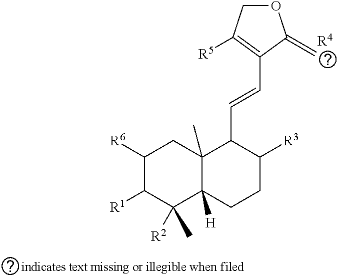
wherein,
R¹ and R² may be selected from a hydroxyl group, a methoxy group, a methylene group, or an ether or ester linked sugar group; hydrogen, substituted or unsubstituted, linear or branched (C₁-C₈) alkyl group such as methyl, ethyl, n-propyl, iso-propyl and the like; aryl group such as phenyl, naphthyl and the like, the aryl group may be substituted; heteroaryl group such as pyridyl, furyl, thiophenyl and the like, the heteroaryl group may be substituted; aralkyl such as benzyl, phenethyl and the like, the aralkyl group may be substituted; heteroaralkyl group such as pyridylmethyl, pyridylethyl, furanmethyl, faranethyl and the like, the heteroaralkyl group may be substituted; (C₂-C₈) alkanoyl group such as ethanoyl, propanoyl, butanoyl and the like, the (C₂-C₈) alkanoyl group may be substituted; (C₃-C₈) alkenoyl group such as propenoyl, butenoyl, pentenyl and the like, (C₃-C₈) alkenoyl group may be substituted; aroyl group such as benzoyl and the like, the aroyl group may be substituted; heteroaroyl group such as pyridyl carbonyl, furyl carbonyl and the like; the heteroaroyl group may be substituted; aralkenoyl group such as phenylpropenoyl, phenylbutenoyl, phenylpentenoyl and the like, the aralkenoyl group may be substituted; aralkanoyl group such as phenylpropanoyl, phenylbutanoyl, phenylpentanoyl and the like, the aralkanoyl group may be substituted; sulfonyl group such as methanesulfonyl, benzenesulfonyl, p-toluenesulfonyl and the like, the sulfonyl group may be substituted.
R³ is selected from a methyl group or a methylene group;
R⁴ is selected from a hydroxyl group or a carbonyl group;
R⁵ is selected from one of the following: a hydroxyl group, an alkyl group, a methoxy group, a methylene group, or an ether or ester linked sugar group; for use in treating an airway disorder.

17. The compound of claim 16 wherein the airway disorder is asthma.

18. The compound of claim 16 wherein the airway disorder is COPD.

19. The compound of claim 16 wherein formula I is Andrographolide.

20. The compound of claim 16 wherein formula I is 14-deoxy-1 1,12-didehydroandrographolide.

21. A Composition comprising a corticosteroid and formula I 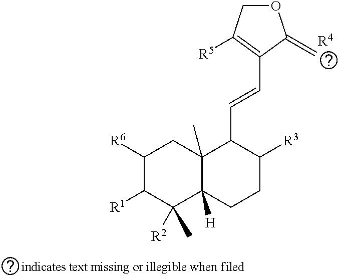
wherein,
R¹ and R² may be selected from a hydroxyl group, a methoxy group, a methylene group, or an ether or ester linked sugar group; hydrogen, substituted or unsubstituted, linear or branched (C₁-C₈) alkyl group such as methyl, ethyl, n-propyl, iso-propyl and the like; aryl group such as phenyl, naphthyl and the like, the aryl group may be substituted; heteroaryl group such as pyridyl, furyl, thiophenyl and the like, the heteroaryl group may be substituted; aralkyl such as benzyl, phenethyl and the like, the aralkyl group may be substituted; heteroaralkyl group such as pyridylmethyl, pyridylethyl, furanmethyl, faranethyl and the like, the heteroaralkyl group may be substituted; (C₂-C₈) alkanoyl group such as ethanoyl, propanoyl, butanoyl and the like, the (C₂-C₈) alkanoyl group may be substituted; (C₃-C₈) alkenoyl group such as propenyl, butenoyl, pentenyl and the like, (C₃-C₈) alkenoyl group may be substituted; aroyl group such as benzoyl and the like, the aroyl group may be substituted; heteroaroyl group such as pyridyl carbonyl, furyl carbonyl and the like; the heteroaroyl group may be substituted; aralkenoyl group such as phenylpropenoyl, phenylbutenoyl, phenylpentenoyl and the like, the aralkenoyl group may be substituted; aralkanoyl group such as phenylpropanoyl, phenylbutanoyl, phenylpentanoyl and the like, the aralkanoyl group may be substituted; sulfonyl group such as methanesulfonyl, benzenesulfonyl, p-toluenesulfonyl and the like, the sulfonyl group may be substituted.
R³ is selected from a methyl group or a methylene group;
R⁴ is selected from a hydroxyl group or a carbonyl group;
R⁵ is selected from one of the following: a hydroxyl group, an alkyl group, a methoxy group, a methylene group, or an ether or ester linked sugar group.

22. The Composition of claim 21 wherein formula I is Andrographolide.

23. The Composition of claim 21 wherein formula I is 14-deoxy-1 1,12-didehydroandrographolide.

24. The Composition of any one of claims 21 to 23 wherein the corticosteroid comprises Dexamethasone, Budesonide, Fluticasone, Ciclesonide, or Beclomethasone Dipropionate.

25. The Composition of any one of claims 21 to 24 for use in treating airway disorders.

26. The composition of claim 25 wherein the airway disorder is asthma.

27. The composition of claim 25 wherein the airway disorder is COPD.
